(19) United States
(12) Patent Application Publication
(10) Pub. No.: US 2022/0005584 A1
(43) Pub. Date: Jan. 6, 2022

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventors: Riuma Takahashi, Tokyo (JP); Ritsuya Tomita, Kanagawa (JP)

(21) Appl. No.: 17/480,739

(22) Filed: Sep. 21, 2021

Related U.S. Application Data
(63) Continuation of application No. PCT/JP2019/051072, filed on Dec. 26, 2019.

(30) Foreign Application Priority Data
Mar. 29, 2019 (JP) 2019-068893
Oct. 3, 2019 (JP) 2019-183352
Dec. 5, 2019 (JP) 2019-220765

(51) Int. Cl. G16H 30/20; G06K 9/66; G16H 15/00; G16H 30/40; G16H 50/20; G16H 50/70; A61B 3/12; A61B 3/14; A61B 3/10

(52) U.S. Cl. CPC G16H 30/20; G06K 9/66; G16H 15/00; G16H 30/40; A61B 3/102; G16H 50/70; A61B 3/1225; A61B 3/14; G16H 50/20

(57) ABSTRACT

An information processing apparatus includes a storage unit configured to store information individually set for each of a plurality of different types of imaging as transmission settings for a plurality of pieces of imaging data obtained by the plurality of different types of imaging, and a transmission unit configured to transmit imaging data on a test subject based on the stored information, the imaging data being obtained by any one of the plurality of different types of imaging.

### CROSS-REFERENCE TO RELATED APPLICATIONS

This application is a Continuation of International Patent Application No. PCT/JP2019/051072, filed Dec. 26, 2019, which claims the benefit of Japanese Patent Applications No. 2019-068893, filed Mar. 29, 2019, No. 2019-183352, filed Oct. 3, 2019, and No. 2019-220765, filed Dec. 5, 2019, all of which are hereby incorporated by reference herein in their entirety.

### BACKGROUND OF THE INVENTION

### Field of the Invention

The present invention relates to an information processing apparatus, an information processing method, and a storage medium.

### Background Art

An ophthalmologic imaging device including a fundus camera and an optical coherence tomography (OCT) configured such that their two optical systems share an optical path in part has heretofore been known (Patent Literature 1).

### CITATION LIST

### Patent Literature

PTL 1: Japanese Patent Laid-Open No. 2011-4978

Conventionally, an information processing apparatus to which an imaging device including a plurality of optical systems for performing different types of imaging is communicably connected or an information processing apparatus inside such an imaging device can make settings related to the difference types of imaging, settings related to imaging data, etc. An information processing apparatus to which at least one of a plurality of imaging devices for performing different types of imaging, such as a fundus camera and an optical coherence tomography (OCT) device, is communicably connectable can also make the foregoing various types of settings and the like. Some such information processing apparatuses can only collectively make settings related to a plurality of different types of imaging data, and improved examiner convenience is desired.

### SUMMARY OF THE INVENTION

One of the objects of the disclosed technique is to enable individual settings related to different types of imaging data. The foregoing object is not restrictive, and it can be regarded as another object of the present application to provide operations and effects that are derived from the configurations discussed in the mode for carrying out the invention to be described below and not achievable by conventional techniques.

According to an aspect of the present disclosure, an information processing apparatus includes a storage unit configured to store information individually set for each of a plurality of different types of imaging as transmission settings for a plurality of pieces of imaging data obtained by the plurality of different types of imaging, and a transmission unit configured to transmit imaging data on a test subject based on the stored information, the imaging data being obtained by any one of the plurality of different types of imaging.

Further features of the present invention will become apparent from the following description of exemplary embodiments with reference to the attached drawings.

### BRIEF DESCRIPTION OF THE DRAWINGS

FIG. 1 is a diagram schematically illustrating a configuration of an ophthalmologic imaging system according to a first exemplary embodiment.

FIG. 2 is a diagram illustrating an example of an automatic transfer setting screen of the ophthalmologic imaging system according to the first exemplary embodiment.

FIG. 3 is a diagram illustrating an example of an imaging screen of the ophthalmologic imaging system according to the first exemplary embodiment.

FIG. 4 is a diagram illustrating an example of an imaging screen of the ophthalmologic imaging system according to the first exemplary embodiment.

FIG. 5 is a diagram illustrating an example of a flowchart of an operation of the ophthalmologic imaging system according to the first exemplary embodiment.

FIG. 6A is a diagram illustrating an example of a report screen displayed on a display unit according to a second exemplary embodiment.

FIG. 6B is a diagram illustrating an example of a report screen displayed on the display unit according to the second exemplary embodiment.

FIG. 7 is a diagram illustrating an example of image quality enhancement processing according to the second exemplary embodiment.

FIG. 8 is a diagram illustrating an example of a user interface according to the second exemplary embodiment.

FIG. 9A is a diagram illustrating an example of a configuration of a neural network used as a machine learning engine according to a sixth modification.

FIG. 9B is a diagram illustrating an example of a configuration of a neural network used as a machine learning engine according to the sixth modification.

FIG. 10A is a diagram illustrating an example of a configuration of a neural network used as a machine learning engine according to the sixth modification.

FIG. 10B is a diagram illustrating an example of a configuration of a neural network used as a machine learning engine according to the sixth modification.

### DESCRIPTION OF THE EMBODIMENTS

Exemplary embodiments for carrying out the present invention will be described in detail below with reference to the drawings. Note that dimensions, materials, shapes, relative positions of components, and the like described in the following exemplary embodiments are freely selectable and can be modified based on the configuration of the apparatuses to which the present invention is applied or various conditions. In the drawings, the same reference numerals are used in difference drawings to denote the same or functionally similar elements. In the drawings, some of the components, members, and processes not important in terms of description can be omitted.

### First Exemplary Embodiment

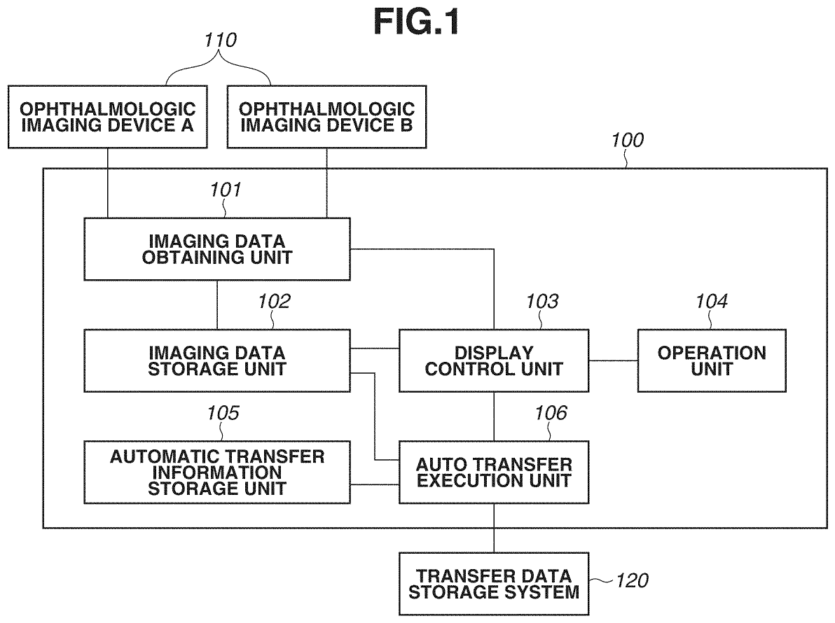

An ophthalmologic imaging system 100 according to the present exemplary embodiment, which is an example of an information processing apparatus, will be described with reference to FIGS. 1 to 5. The ophthalmologic imaging system 100 according to the present exemplary embodiment can convert, into a previously-set data format, that of imaging data depending on the ophthalmologic imaging device capturing the image, and automatically transfer the data. The following description will be given by using a fundus camera and an optical coherence tomography (OCT) as examples of ophthalmologic devices to be handled.

### (System Configuration)

A system configuration will be described with reference to FIG. 1. FIG. 1 schematically illustrates a configuration of the ophthalmologic imaging system 100. The ophthalmologic imaging system 100 includes an imaging data obtaining unit 101, an imaging data storage unit 102, a display control unit 103, an operation unit 104, an automatic transfer information storage unit 105, and an automatic transfer execution unit 106. As employed in the present exemplary embodiment and the like, “transfer” may refer to a case where an imaging signal (for example, OCT interference signal) from an imaging device is simply transmitted as imaging data, for example. As employed in the present exemplary embodiment and the like, “transfer” may also refer to a case where imaging data processed as an image generated from an imaging signal is transmitted. As employed in the present exemplary embodiment and the like, “transfer” may also refer to a case where data processed as a report image corresponding to a report screen including an image generated from an imaging signal is transmitted as imaging data. Examples of the image generated from an imaging signal may include at least one tomographic image (B-scan image) and a front image (en-face image) obtained by using at least part of a depth range of a plurality of pieces of tomographic image data (three-dimensional tomographic image data, volume data) obtained at difference positions. The information processing apparatus may be configured such that the depth range can be set based on the examiner's instructions. Here, the information processing apparatus may be configured such that the depth range is set by changing the positions of layer boundaries obtained by segmentation processing on a tomographic image on the tomographic image based on the examiner's instructions. As employed in the present exemplary embodiment and the like, “automatic transfer” does not mean that the examiner's instructions will not be used at all as a trigger to start transmitting imaging data. For example, if the transmission setting of the imaging is automatically set, it is intended that another instruction given by the examiner and not originally intended to start transmitting imaging data (for example, an examination end instruction) also serves as an instruction to start transmitting the imaging data. In other words, “automatic transfer” employed in the present exemplary embodiment and the like may refer to any case where another instruction not directly intended to start transmitting imaging data is configured to also serve as an instruction to start transmitting the imaging data.

The imaging data obtaining unit 101 can receive captured imaging data from at least two or more ophthalmologic imaging devices 110. In the present exemplary embodiment, the imaging data obtaining unit 101 can receive fundus images and retinal tomographic data from a fundus camera and an optical coherence tomography device, respectively. Here, while the plurality of imaging devices for performing different types of imaging can be communicably connected to the information processing apparatus at the same time, the present exemplary embodiment and the like are applicable if at least one is connected. More specifically, the information processing apparatus is configured such that transmission settings for the pieces of imaging data from the plurality of imaging devices can be individually made in a situation where any one of the imaging devices is communicably connected to the information processing apparatus. The information processing apparatus according to the present exemplary embodiment may be an information processing apparatus to which an imaging device including a plurality of optical systems for performing different types of imaging is communicably connected, or an information processing apparatus inside such an imaging device. The information processing apparatus according to the present exemplary embodiment may be a personal computer, for example. A desktop personal computer (PC), a laptop PC, or a tablet PC (portable information terminal) may be used.

The imaging data storage unit 102 receives and records imaging data obtained by the imaging data obtaining unit 101. Here, additional information about the imaging data, including patient information such as the name, date of birth, sex, patient identifier (ID), and race data of the patient (examinee), examination information such as the date and time of examination, a reception number, and an examination identification ID, and imaging information such as an imaging time, an imaging mode name, imaging parameters, a device name, and an imaging determination, is additionally registered.

The display control unit 103 displays an imaging operation screen of the ophthalmologic imaging devices 110 in obtaining imaging data and a check screen for displaying imaging data obtained by the imaging data obtaining unit 101 and recorded in the imaging data storage unit 102 on a non-illustrated monitor that is an example of a display unit.

The operation unit 104 can run imaging by the ophthalmologic imaging devices 110 on the imaging operation screen and select an imaging success/failure determination on imaging data displayed on an imaging result check screen via a mouse and a keyboard. For the imaging success/failure determination on the imaging data, imaging determination information can be input by an operator checking the imaging data displayed on the imaging result check screen and pressing a success or failure button displayed on the screen, for example. Moreover, automatic transfer (automatic transmission) can be started via the operation unit 104. For example, a transition from the imaging operation screen to another screen can be used as an automatic transfer start trigger. Pressing of an examination completion button displayed on the imaging operation screen can be used as an automatic transfer start trigger. The display unit may be a touch panel display, in which case the display unit is also used as the operation unit 104.

The automatic transfer information storage unit 105 stores settings prepared in advance to automatically transfer imaging data. The stored settings include ophthalmologic devices targeted for automatic transfer, an automatic transfer destination, and the data format of imaging data to be transferred.

The automatic transfer execution unit 106 receives the automatic transfer start trigger instructed from the operation unit 104 via the display control unit 103, and transfers imaging data to a transfer data storage system 120 serving as an automatic transfer destination based on automatic transfer information obtained from the automatic transfer information storage unit 105. Here, the automatic transfer execution unit 106 checks the imaging data of which imaging device the automatic transfer information is targeted for, converts, into a data format specified by the automatic transfer information, that of the corresponding imaging data, and transfers the converted data to the transfer data storage system 120.

### (Automatic Transfer Information According to Present Exemplary Embodiment)

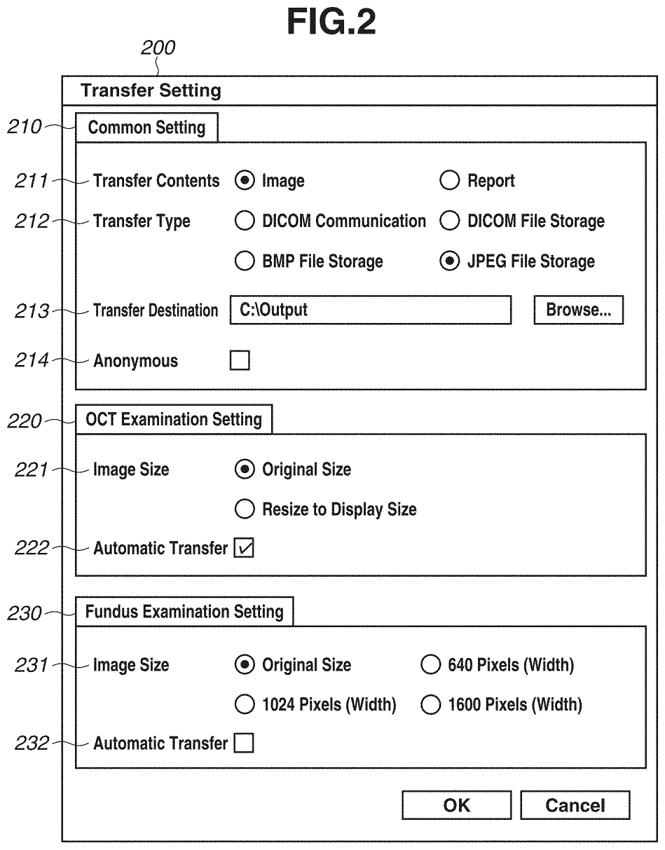

Next, the automatic transfer information stored in the automatic transfer information storage unit 105 will be described with reference to FIG. 2. FIG. 2 illustrates an example of a screen for setting the automatic transfer (automatic transmission) contents according to the present exemplary embodiment. This screen is one for making an automatic transfer setting. In cases such as when the settings vary from one imaging device to another and where automatic transfer to a plurality of transfer destinations (transmission destinations) is intended, a plurality of automatic transfer settings can be registered in the automatic transfer information storage unit 105 by separately making the respective settings.

A transfer setting screen 200 includes a common setting area 210, an OCT examination setting area 220, and a fundus examination setting area 230. The OCT examination setting area 220 and the fundus examination setting area 230 are examples of an individual setting area.

The common setting area 210 is an area for setting transfer setting (transmission setting) items common to the OCT examination and the fundus examination, and includes a transfer contents setting 211, a transfer type setting 212, a transfer destination setting 213, and an anonymization setting 214.

The transfer contents setting 211 (transmission contents setting) can select either “Image” or “Report” as data contents to be transmitted to the transfer destination (transmission destination). If “Image” is selected as the transfer contents setting, the images of the imaging data obtained by the ophthalmologic imaging devices 110 imaging the examinee's eye to be examined are set to be transferred. For example, in the case of imaging data obtained by the fundus camera capturing an image of the eye to be examined, a fundus camera image is transferred. In the case of imaging data obtained by the OCT capturing an image of the eye to be examined, at least one tomographic image (B-scan image) is transferred (transmitted). Since OCT imaging can obtain a plurality of tomographic images by one imaging operation, all the plurality of tomographic images then may be set to be transferred. In some cases, OCT imaging can also capture a scanning laser ophthalmoscopy (SLO) or another retinal front image at the same time. The retinal front image then may be set to be transferable together. If the OCT captures a three-dimensional image of the retina, a retinal front image that is a front image reconstructed from the OCT imaging data may be set to be transferable together. Moreover, an image in which a tomographic image and a retinal front image are juxtaposed and the imaging position of the tomographic image is indicated on the retinal front image may be set to be transferred. If “Report” is selected as the transfer contents setting, a report image in which imaging data and related data are arranged in a specific layout, like one displaying a plurality of pieces of imaging data in a row or one displaying pieces of analysis information about the imaging data in a row, is set to be transferred. For example, in the case of the fundus camera, a report image in which a plurality of captured images is arranged in a matrix is transferred. In the case of the OCT, a report image displaying a map image indicating retinal thicknesses at respective positions in a color scale on a retinal front image in addition to tomographic images and the retinal front image may be transferred. A report image may include the patient information, the examination information, and the imaging information registered in the imaging data storage unit 102 as additional information about the imaging data. The options of the transfer contents (transmission contents) are not limited to two-dimensional images like various two-dimensional medical images and report images. For example, OCT three-dimensional structure data may be able to be transferred.

The transfer type setting 212 (transmission type setting) can select the data transfer method and the image format of the contents set by the transfer contents setting 211. For example, if the transfer destination (transmission destination) is a Digital Imaging and Communications in Medicine (DICOM) storage server, DICOM communication is selected. If the transfer destination (transmission destination) is a storage such as a hard disk or a network attached storage (NAS), file storage is selected. In the case of file storage, an image format for storage is selected from among a bitmap, Joint Photography Experts Group (JPEG), and DICOM. In other words, the contents of the imaging data (accessory information etc.) to be transmitted vary depending on the transmission type and the storage format.

The transfer destination setting 213 (transmission destination setting) can set the transfer destination of the imaging data. The method for setting the transfer destination varies depending on whether the data transfer method in the transfer type setting 212 is DICOM communication or file storage, and the input items in the screen changes based on the selection. If DICOM communication is selected, a hostname, a port number, and a server application entity (AE) title needed to communicate with the transfer destination DICOM storage server can be input. In such a case, the present screen may include a function of checking whether communication can be made with the input transfer destination. If file storage is selected, the path of the storage location can be input.

The anonymization setting 214 can select whether to anonymize personal information included in the data to be transferred. If anonymization is set up, personal information such as the patient's name included in a report image, in DICOM or JPEG tag information, and/or in a filename is set to be anonymized and transferred. The anonymization setting may refer to a predetermined method for anonymization. An anonymization setting screen may be provided to make fine settings on the anonymization method, or the anonymization setting may be directly provided on the transfer setting screen.

The OCT examination setting area 220 is an area for setting items to be applied in transferring imaging data captured by the OCT, and includes an OCT examination image size setting 221 and an OCT examination automatic transfer setting 222.

The OCT examination image size setting 221 is an item to be enabled if the transfer contents setting 211 is an image, and can set the image size of the tomographic image to be transferred. Original size is selected to transfer the tomographic image in the same size as the imaging data captured by the OCT. Resize to display size is selected to transfer the tomographic image in a size for the ophthalmologic imaging system 100 to display the tomographic image on the monitor.

The OCT examination automatic transfer setting 222 can select automatic transfer for OCT examination. For example, if automatic transmission is checked, automatic transmission is set to on. In performing automatic transfer, the automatic transfer execution unit 106 automatically transfers the contents set on this transfer setting screen 200 if the ophthalmologic imaging device by which the imaging data is captured is the OCT when the automatic transfer execution unit 106 receives an automatic transfer start trigger.

The fundus examination setting area 230 is an area for setting items to be applied in transferring imaging data captured by the fundus camera, and includes a fundus examination imaging size setting 231 and a fundus examination automatic transfer setting 232.

The fundus examination imaging size setting 231 is an item to be enabled if the transfer contents setting 211 is an image, and can set the image size of the fundus camera image to be transferred. Original size is selected to transfer the fundus camera image in the same size as when captured. To transfer the fundus camera image in a different size, the item for the specific width is selected. In transferring an image having a width less than or equal to the selected size, the image is set to be transferred in its original size.

The fundus examination automatic transfer setting 232 can select automatic transfer (automatic transmission) for fundus examination. For example, if automatic transmission is checked, automatic transmission is set to on. In the case of automatic transfer, the automatic transfer execution unit 106 automatically transfers the contents set on this transfer setting screen 200 if the ophthalmologic imaging device by which the imaging data is captured is the fundus camera when the automatic transfer execution unit 106 receives an automatic transfer start trigger.

### (Imaging Screens According to Present Exemplary Embodiment)

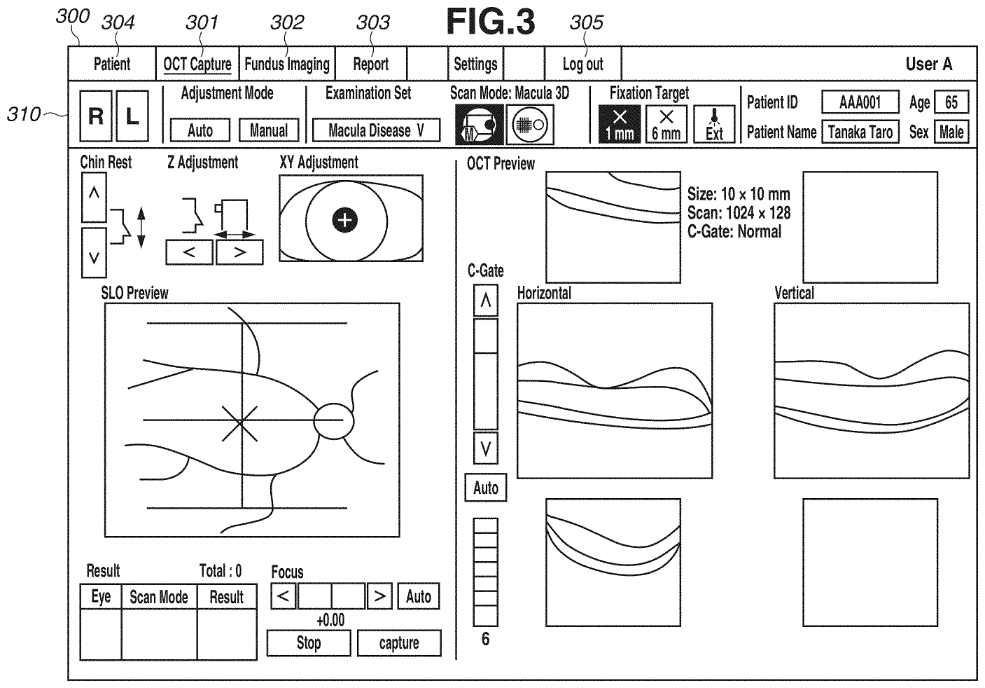

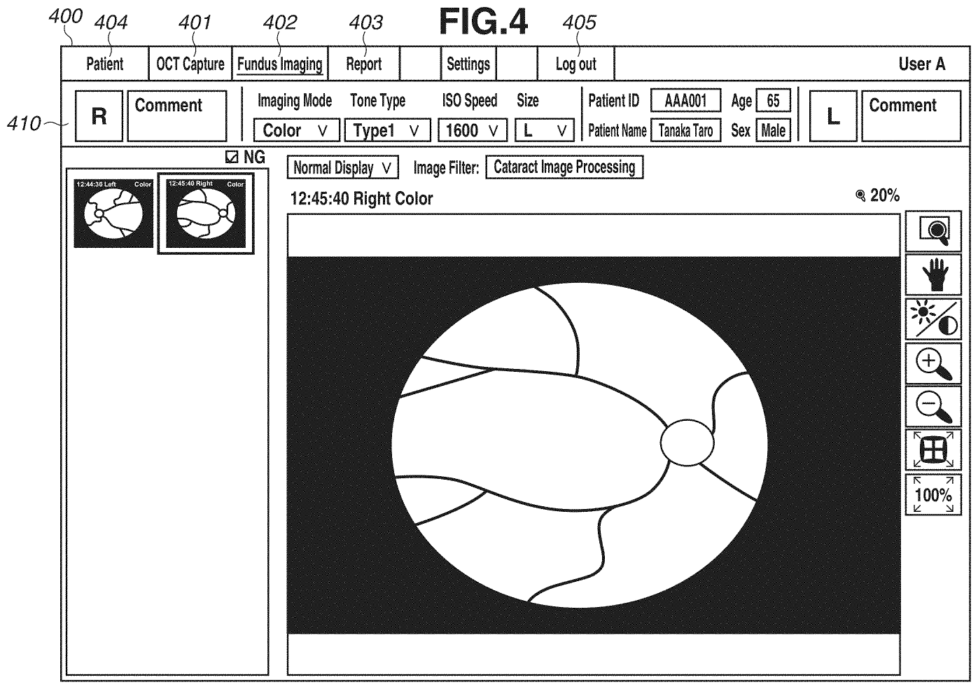

FIGS. 3 and 4 illustrate examples of screens for performing examination using the ophthalmologic imaging devices, displayed by the display control unit 103. The present exemplary embodiment deals with screen examples where a transition from a screen for capturing an image using an ophthalmologic imaging device 110 to another screen is used as an automatic transfer start trigger.

An ophthalmologic imaging system screen 300 can display a plurality of screens, and the screens can be switched in a tab manner. FIG. 3 illustrates an example of a screen for performing OCT imaging, which is displayed when an OCT imaging tab 301 is selected. FIG. 4 illustrates an example of a screen for performing fundus camera imaging, which is displayed when a fundus imaging tab 402 is selected. If the OCT imaging tab 301 is selected, an OCT imaging screen 310 is displayed in the tab, where OCT imaging can be performed and an imaging result can be displayed. Here, FIG. 3 illustrates a preview screen, where a moving image of the anterior eye part is displayed in an upper left display area, an SLO moving image of the fundus is displayed in a lower left display area, and an OCT tomographic moving image is displayed in a right display area. Here, the information processing apparatus may be configured to switch to a display of a non-illustrated imaging confirmation screen if various optical adjustments of an alignment, focus, coherence gate, and the like are made and OCT imaging is executed on the preview screen. The information processing apparatus may be configured to switch to a display of the preview screen if OCT imaging is OK on the imaging confirmation screen. If the fundus imaging tab 302 is selected, a fundus camera imaging screen 410 is displayed in the tab, where an image captured by the fundus camera can be displayed.

Tabs other than for the imaging screens include a report tab 303 displaying a report screen where imaging data on the imaged patient is displayed, and a patient tab 304 displaying a patient screen for creating and selecting a record of a patient to start examination on another patient or display imaging data on another patient. Here, the report screen may be configured to be switchable between various display screens, including a display screen for follow-up and a three-dimensional volume rendering display screen. The report screen may be configured such that one of the foregoing various display screens can be set as its initial display screen. The report screen may be configured such that not only the initial display screen but also the presence or absence of image quality enhancement processing, the presence or absence of analysis result display, a depth range for generating a front image, and the like can be set as its initial display. If “Report” is selected as the foregoing transmission contents, a report image generated based on the contents set as the initial display of the report screen may be transmitted. The report screen may be a display screen that is used in use cases such as where OCT imaging is performed after fundus imaging, and that displays fundus images, OCT images, and the like together. Suppose that, for example, information where the transmission of a report image is set to on as a transmission setting is stored, and image quality enhancement processing is on as a setting on the initial display of the report image. In such a case, a transmission unit can transmit, as imaging data, a report image corresponding to a report screen displaying medical images obtained by the image quality enhancement processing.

Furthermore, there is a logout button 305 to log out to end using the ophthalmologic imaging system and display a login screen. Automatic transfer is started if the tabs other than for the imaging screens or the logout button 305 is selected. Since automatic transfer is not performed upon transition from the OCT imaging tab 301 to the fundus imaging tab 302 or from the fundus imaging tab 302 to the OCT imaging tab 301, the operation here is to automatically transfer both the imaging data of the fundus imaging and the imaging data of the OCT imaging in a collective manner after the execution of both the fundus imaging and the OCT imaging. Not performing automatic transfer between the imaging tabs can prevent the operator's imaging operations from being hindered by the automatic transfer processing. For example, in a case where the automatic transfer processing is performed in parallel with screen operations, imaging operations can be prevented from a processing failure due to the load of the automatic transfer processing. In a case where the automatic transfer processing is performed not in parallel with screen operations but completed before the next imaging screen is displayed, the patient can be prevented from being kept waiting by the automatic transfer processing during a series of imaging operations on the patient. Note that if the automatic transfer processing is performed in parallel with screen operations and the system has sufficient performance, the transition from the OCT imaging tab 301 to the fundus imaging tab 302 and the transition from the fundus imaging tab 302 to the OCT imaging tab 301 may also be handled as automatic transfer start triggers. In such a case, the imaging data of a single ophthalmologic imaging device is always automatically transmitted. Only the patient tab 304 and the logout button 305 may be handled as automatic transfer start triggers without handling the transition to the report screen as an automatic transfer start trigger, so that automatic transfer is performed in units of transitions from one patient to another. In such a case, information added and edited in the report screen can also be handled as information to be added upon automatic transfer.

### (Automatic Transfer Processing Flow According to Present Exemplary Embodiment)

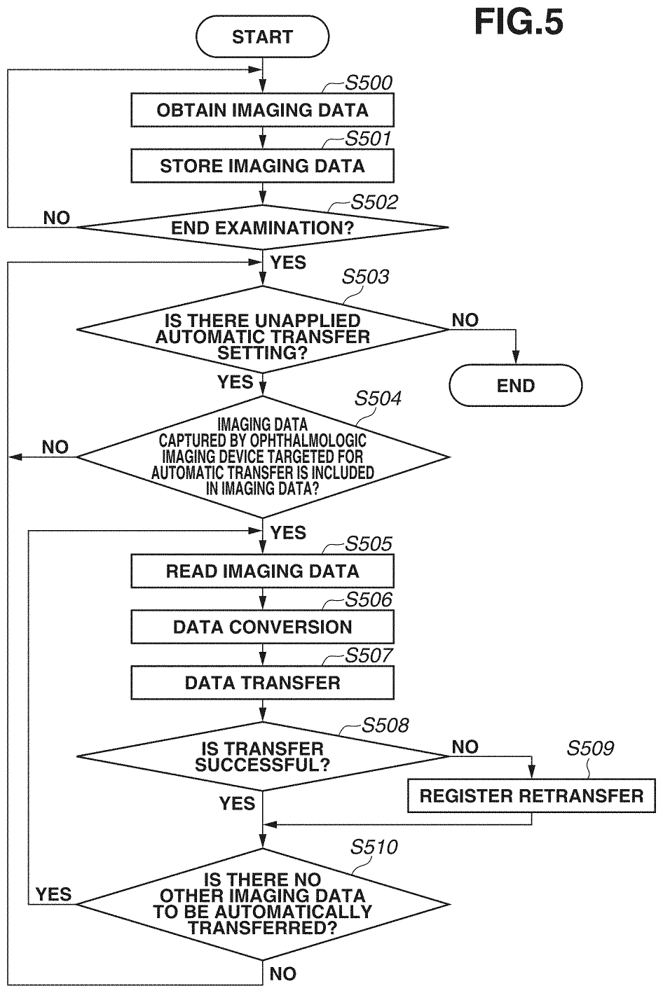

Next, a flow of the automatic transfer processing according to the present exemplary embodiment will be described with reference to FIG. 5. FIG. 5 is a flowchart of the operation of the automatic transfer processing according to the present exemplary embodiment.

The ophthalmologic imaging devices targeted for automatic transfer can vary depending on the transfer settings. In executing automatic transfer, the automatic transfer execution unit 106 thus checks the automatic transfer settings in the automatic transfer information storage unit 105 and the ophthalmologic imaging devices 110 targeted for automatic transfer stored in the imaging data storage unit 102, and executes automatic transfer to the transfer data storage system 120 only if the ophthalmologic imaging devices 110 are targeted for automatic transfer.

Specifically, in step S500, the operator performs imaging using an ophthalmologic imaging device 110, and the imaging data obtaining unit 101 obtains imaging data from the imaging device 110. Here, the imaging data obtaining unit displays the imaging data via the display control unit 103, and the operator enters whether the imaging is successful or failed.

In step S501, the imaging data obtaining unit 101 stores the imaging data obtained in step S500 and the result about whether the imaging is successful or failure, entered by the operator, into the imaging data storage unit 102 along with additional information. If an imaging failure is entered, the imaging data may be either not stored or stored in a location other than the imaging data storage unit 102.

In step S502, if the operator continues examination, imaging is performed and the processing returns to step S500. If the operator selects (presses) tabs other than the imaging tabs such as the OCT imaging tab 301 and the fundus imaging tab 302 on the ophthalmologic imaging system screen 300, the imaging screen is switched to another display screen. Here, the examination is determined to have ended, and an automatic transfer start trigger is transmitted to the automatic transfer execution unit 106. Suppose, for example, that the fundus imaging has ended and the OCT imaging tab is pressed. Processing during an OCT preview (for example, processing intended for a moving image of the anterior eye part, an SLO moving image of the fundus, an OCT tomographic moving image, and various optical adjustments) is high in load. If the fundus imaging data is transmitted during the OCT preview, the processing during the OCT preview can thus fail. The information processing apparatus may therefore be configured not to perform automatic transfer not only when the current imaging tab is selected but also when the other imaging tab is selected. However, in the present exemplary embodiment and the like, the information processing apparatus may be configured to perform automatic transfer when the imaging tab other than the currently selected one is selected. The information processing apparatus may also be configured to switch the display screen to a login screen and determine that the examination has ended if the logout button 305 is selected while an imaging screen is displayed. In such a case, the selection of the logout button 305 can be used as an automatic transfer start trigger.

In step S503, the automatic transfer execution unit 106 reads the transfer settings from the automatic transfer information storage unit 105 one by one, and determines whether there is an automatic transfer setting. If there is an automatic transfer setting, the processing proceeds to step S504. If there is no automatic transfer setting, the automatic transfer execution unit 106 ends the automatic transfer processing. If the information processing apparatus is configured such that only one transfer setting can be registered, step S503 and the subsequent steps are not indispensable. As described above, a plurality of patterns of transfer settings may be made registrable. In such a case, if there is registered a plurality of patterns of transfer settings, then in step S503, information (data) corresponding to the settings of the plurality of patterns of transfer settings may be transmitted in order. For example, even if a first transfer setting and a second transfer setting are contradictory to each other, the data on these settings may be transmitted in order.

In step S504, the automatic transfer execution unit 106 checks the ophthalmologic imaging device targeted for automatic transfer in the automatic transfer setting checked in step S503 and the ophthalmologic imaging device that has captured the imaging data stored in step S501, and determines whether imaging data captured by the ophthalmologic imaging device targeted for automatic transfer is included in the imaging data. The automatic transfer execution unit 106 checks the settings of the OCT examination automatic transfer setting 222 and the fundus examination automatic transfer setting 232 in the transfer setting screen 200 for the ophthalmologic imaging device targeted for automatic transfer. If imaging data captured by the ophthalmologic imaging device targeted for automatic transfer is included in the imaging data, the processing proceeds to step S505 to enter automatic transfer processing. If there is no imaging data captured by the ophthalmologic imaging device targeted for automatic transfer, the processing returns to step S503 to check the presence or absence of a next automatic transfer setting.

In step S505, the automatic transfer execution unit 106 sequentially reads the imaging data of the ophthalmologic imaging device targeted for automatic transfer from the imaging data stored in the imaging data storage unit 102.

In step S506, the automatic transfer execution unit 106 performs data conversion on the imaging data read in step S505 based on the transfer settings. For example, if the imaging data is that of the fundus camera, and the image is selected in the transfer contents setting 211 and 1600 pixels (width) is selected in the image size setting 231, the automatic transfer execution unit 106 converts the image captured by the fundus camera into image information having a width of 1600 pixels. If the transfer type setting 212 is the JPEG file storage, the automatic transfer execution unit 106 further performs data conversion into a JPEG format, and adds patient information, examination information, and imaging information into the JPEG tag. Here, if the anonymization setting 214 is set, the automatic transfer execution unit 106 anonymizes personal information in the information included in the JPEG tag and adds the resultant.

In step S507, the automatic transfer execution unit 106 transfers the data converted in step S506 to the transfer destination set in the transfer destination setting 213. If the file storage is selected in the transfer type setting 212, the automatic transfer execution unit 106 stores the file into the specified path. If DICOM communication is selected, the automatic transfer execution unit 106 transfers the data to the transfer data storage system that is the transfer destination.

In step S508, the automatic transfer execution unit 106 checks the result of the data transfer executed in step S507. If the transfer is normally completed, the processing proceeds to step S510. If the transfer fails, the processing proceeds to step S509 to perform retransfer processing.

In step S509, the automatic transfer execution unit 106 records the transfer-failed data as a retransfer target. For example, if the storage location does not have sufficient capacity or if the storage location or the communication destination is inaccessible due to a network failure, the automatic transfer execution unit 106 registers a setting for automatically executing retransfer upon next login or after a lapse of a certain time. The retransfer may be not automatically but manually executed by the operator.

In step S510, the automatic transfer execution unit 106 checks whether there is no other imaging data to be a candidate for the automatic transfer setting checked in step S503. If there is such imaging data, the processing proceeds to step S505 to automatically transfer the next imaging data. If there is no such imaging data, the processing proceeds to step S503 to check whether there is another automatic transfer setting. Here, if, in step S503, all automatic transfers have been completed, automatic transfer is ended. Here, the automatic transfer execution unit 106 may make a notification of the result of the automatic transfer. Whether all the imaging data targeted for all the automatic transfer settings has been successfully automatically transferred is notified as the notification content. The automatic transfer execution unit 106 may notify the operator of information about the imaging data registered for retransfer in step S509.

In the processing of step S503 and the subsequent steps in this automatic transfer processing flow, the automatic transfer processing may be performed separate from and in parallel with the screen display by the display control unit 103 from start to end, and the operator can make screen operations. Alternatively, a transfer in progress message may be displayed and presented on the screen such that no screen operation will be made until the completion of the automatic transfer processing. If the automatic transfer processing is performed in parallel with the screen display, the notification of the automatic transfer result after the completion of the automatic transfer processing may be made by preparing a result display area on the screen and displaying the result at the end of the automatic transfer. Details of the automatic transfer result may be displayed by the user then selecting the result display area.

According to the present exemplary embodiment described above, in automatically transferring captured imaging data, the ophthalmologic imaging system 100 can perform automatic transfer while modifying the transfer contents based on the type of ophthalmologic imaging device capturing the imaging data. This is extremely favorable since only imaging data needed for the ophthalmologic imaging devices is appropriately transferred to the automatic transfer destination.

In the present exemplary embodiment, automatic transfer is started when imaging by the ophthalmologic imaging device is completed and the screen transitions to a different screen. However, automatic transfer may be started upon each imaging operation. In such a case, after the storage of the imaging data in step S501, the processing proceeds to step S503 to perform automatic transfer. In the case of automatically transferring a report image and the like where a plurality of pieces of imaging data is needed, the operation here is to perform the automatic transfer when all the needed imaging data is determined to have been obtained. In the present exemplary embodiment, imaging data obtained from each of the two devices, the fundus camera and the OCT, is automatically transferred. However, this is not restrictive. If a single device has the functions of a plurality of ophthalmologic imaging devices such as a fundus camera and an OCT, automatic transfer can be similarly appropriately performed on each ophthalmologic imaging device function by checking which ophthalmologic imaging device function each piece of imaging data is obtained by and automatically transferring the imaging data obtained by the function of the ophthalmologic imaging device targeted for automatic transfer. Moreover, in the present exemplary embodiment, automatic transfer is started in response to an automatic transfer start trigger after execution of imaging. However, this is not restrictive. A button for collective transfer may be prepared on a screen other than the imaging screens, and transfer may be started at any timing selected by the operator and performed depending on the ophthalmologic imaging devices targeted for automatic transfer. In such a case, information about the automatic transfer settings and the imaging data to be automatically transferred may be presented to the operator. The operator may be permitted to change the target automatic transfer settings and the range of imaging data and start automatic transfer.

### Second Exemplary Embodiment

An information processing apparatus according to the present exemplary embodiment includes an image quality enhancement unit (not illustrated) for applying machine learning-based image quality enhancement processing as image quality enhancement means for enhancing the image quality of motion contrast data. Here, the image quality enhancement unit of the information processing apparatus generates a motion contrast image having high image quality (low noise and high contrast) equivalent to that of a motion contrast image generated from a large number of tomographic images, by inputting a low image quality motion contrast image generated from a small number of tomographic images into a machine learning model. As employed herein, a machine learning model refers to a function generated by performing machine learning with training data including pairs of input data that is a low image quality image assumed as a processing target and obtained under a predetermined imaging condition and output data (ground truth data) that is a high image quality image corresponding to the input data. The predetermined imaging condition includes an imaging region, an imaging method, an imaging angle of view, and an image size.

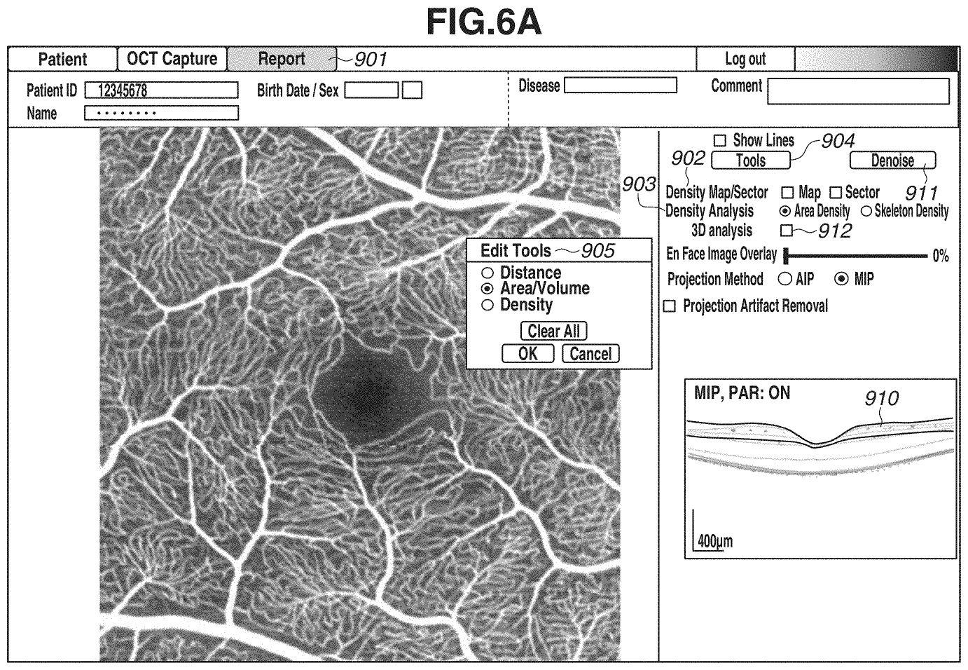

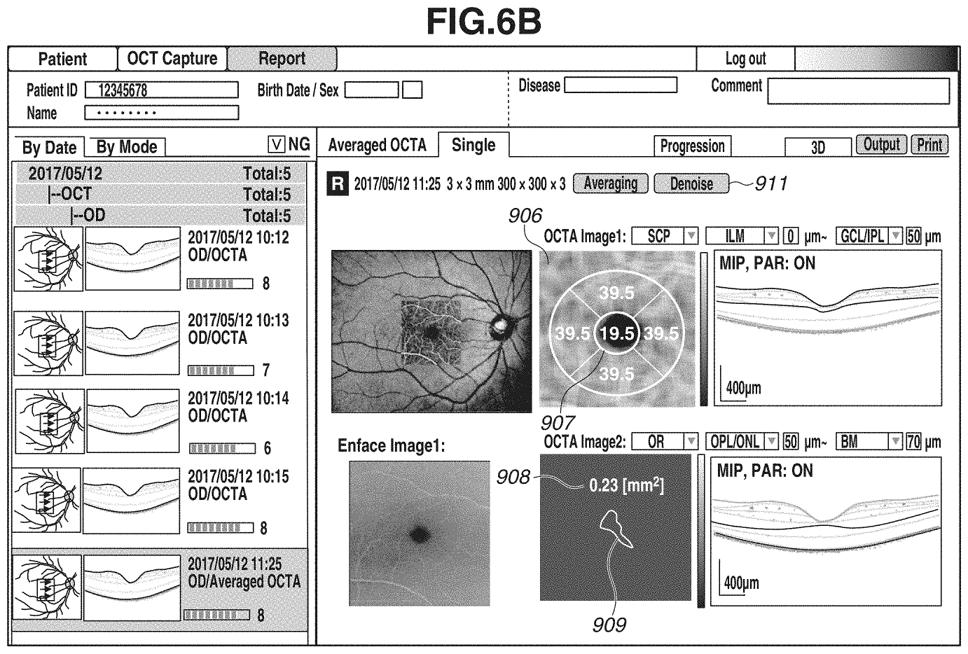

For example, a low image quality motion contrast image is obtained by the following manner. The operator initially operates an operation unit 104 to press an imaging start (Capture) button in an imaging screen (preview screen), whereby optical coherence tomography angiography (OCTA) imaging is started under a set imaging condition in response to the operator's instruction. Here, a control unit (not-illustrated) of the information processing apparatus instructs an optical coherence tomography (OCT) to perform OCTA imaging based on settings specified by the operator, and obtains OCT tomographic images supported by the OCT. The OCT also obtains an SLO image by using an SLO optical system on an optical path separated using a dichroic mirror that is an example of a wavelength separation member, and performs tracking processing based on the SLO moving image. Here, the imaging condition is set, for example, by 1) registering a macular disease examination set, 2) selecting an OCTA scan mode, and 3) setting the following imaging parameters and the like. Examples of the set imaging parameters include 3-1) a scan pattern: 300 A-scans×300 B-scans, 3-2) a scan area size: 3×3 mm, and 3-3) a main scanning direction: horizontal direction. Examples of the set imaging parameters further include 3-4) a scan spacing: 0.01 mm, 3-5) a fixation lamp position: macular (fovea), 3-6) the number of B-scans per cluster: 4, 3-7) a coherence gate position: a vitreous body side, and 3-8) a predetermined display report type: a single eye examination report. The imaging data obtaining unit 101 generates a motion contrast image (motion contrast data) based on the obtained OCT tomographic images. Here, after the generation of the motion contrast image, a non-illustrated correction unit may perform processing for reducing projection artifacts occurring on the motion contrast image. A display control unit 103 then displays generated tomographic images, a three-dimensional motion contrast image, a motion contrast front image, information about the imaging condition, and the like on a display unit (not illustrated). Here, in the present exemplary embodiment, the image quality enhancement unit performs the image quality enhancement processing on the motion contrast image by the user pressing a button 911 (an example of an image quality enhancement button) displayed on the upper right of a report screen in FIG. 6A. In other words, the image quality enhancement button is a button for giving an instruction to execute the image quality enhancement processing. It will be understood that the image quality enhancement button may be a button for giving an instruction to display a high image quality image (generated before the pressing of the image quality enhancement button).

In the present exemplary embodiment, the input data used as the training data is low image quality motion contrast images generated from a single cluster with a small number of tomographic images. The output data (ground truth data) used as the training data is high image quality motion contrast images obtained by addition average of a plurality of pieces of aligned motion contrast data. Note that the output data to be used as the training data is not limited thereto. For example, high image quality motion contrast images generated from a single cluster including a large number of tomographic images may be used. The output data to be used as the training data may be high image quality motion contrast images obtained by reducing motion contrast images having higher resolution (higher magnification) than the input images to the same resolution (same magnification) as the input images. The pairs of input and output images to be used in training the machine learning model are not limited to the foregoing, and any combinations of conventional images may be used. For example, images obtained by adding a first noise component to motion contrast images obtained by a ophthalmologic imaging system 100 or other devices may be used as the input images, and images obtained by adding a second noise component (different from the first noise component) to the motion contrast images (obtained by the ophthalmologic imaging system 100 or other devices) may be used as the output images, in training the machine learning model. In other words, any image quality enhancement unit that enhances the image quality of motion contrast data input as an input image by using a trained model for image quality enhancement, obtained by training with training data including motion contrast data on the fundus, may be used.

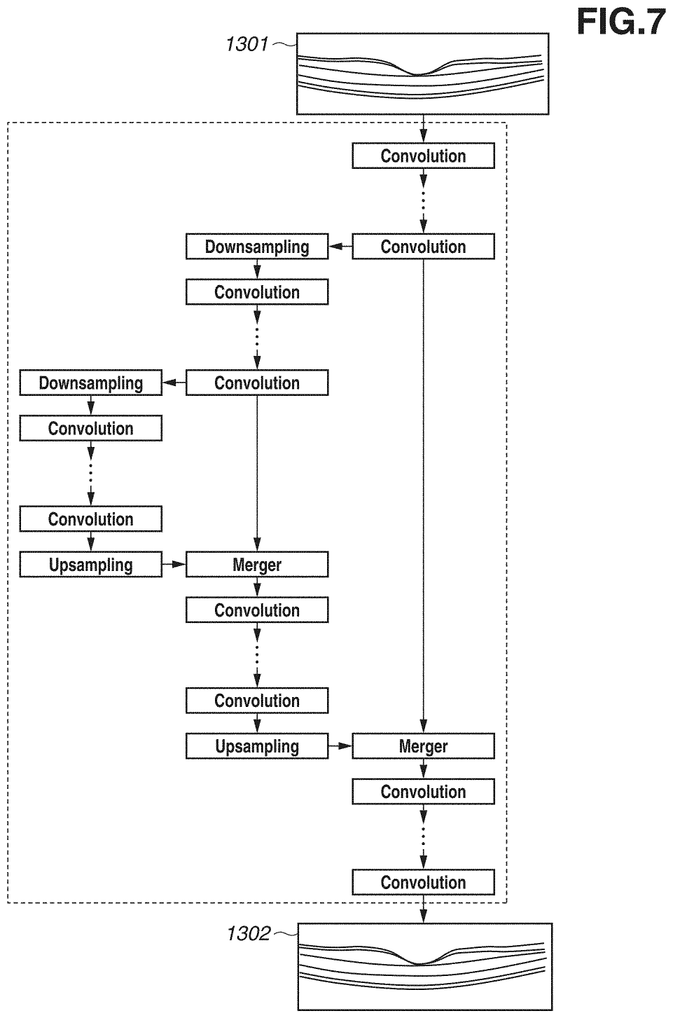

FIG. 7 illustrates a configuration of the machine learning model in the image quality enhancement unit according to the present exemplary embodiment. The machine learning model is a convolutional neural network (CNN), and includes a plurality of layers in charge of processing for processing a group of input values and outputting the resultant. Types of layers included in the foregoing configuration include a convolution layer, a downsampling layer, an upsampling layer, and a merger layer. A convolution layer is a layer for performing convolutional processing on a group of input values based on parameters such as the kernel size of a set filter, the number of filters, a stride value, and a dilation value. The number of dimensions of the kernel size of the filter may be changed based on the number of dimensions of an input image. A downsampling layer is a layer for performing processing for making the number of output values smaller than that of input values by decimating or combining the group of input values. A specific example is max pooling processing. An upsampling layer is a layer for performing processing for making the number of output values greater than that of input values by duplicating the group of input values or adding values interpolated from the group of input values. A specific example includes linear interpolation processing. A merger layer is a layer for performing processing for inputting groups of values, such as a group of output values of a layer and a group of pixel values constituting an image, from a plurality of sources and merging the groups of values by connecting or adding the groups of values. With such a configuration, a group of values output by passing a group of pixel values constituting an input image 1301 through convolutional processing blocks is merged with the group of pixel values constituting the input image 1301 in a merger layer. The merged group of pixel values is then formed into a high image quality image 1302 in the final convolution layer. Although not illustrated in the diagram, a batch normalization layer and/or an activation layer using a rectifier linear function (rectifier linear unit) may be built in or otherwise incorporated after the convolution layer as a modification of the CNN configuration. In FIG. 7, the processing target image is described to be a two-dimensional image for ease of description. However, the present invention is not limited thereto. The present invention also covers a case where a three-dimensional low image quality motion contrast image is input into the image quality enhancement unit to output a three-dimensional high image quality motion contrast image.

Now, a graphics processing unit (GPU) can efficiently perform calculations by processing more pieces of data in parallel. To execute processing using a GPU is thus effective in training a learning model a plurality of times like deep learning. In the present exemplary embodiment, the processing by the information processing apparatus that is an example of a learning unit (not illustrated) thus uses a GPU in addition to a CPU. Specifically, in executing a training program including a learning model, the CPU and the GPU perform training by executing computing in a cooperative manner. The computing in the processing by the training unit may be executed by either the CPU or the GPU alone. Like the training unit, the image quality enhancement unit may also use a GPU. Moreover, the training unit may include an error detection unit and an update unit that are not illustrated. The error detection unit obtains an error between output data output from the output layer of the neural network in response to input data input to the input layer and ground truth data. The error detection unit may calculate the error between the output data from the neural network and the ground truth data by using a loss function. Based on the error obtained by the error detection unit, the update unit updates coupling weight coefficients and the like between nodes of the neural network to reduce the error. The update unit updates the coupling weight coefficients and the like by backpropagation, for example. Backpropagation is a technique for adjusting the coupling weight coefficients and the like between the nodes of the neural network such that the foregoing error decreases.

The operator can give an instruction to start OCTA analysis processing by using the operation unit 104. In the present exemplary embodiment, a motion contrast image of FIG. 6B that is a report screen is double-clicked to transition (screen-transition) to FIG. 6A that is an example of a report screen. The motion contrast image is displayed on an enlarged scale, and analysis processing can be performed using the information processing apparatus. Any type of analysis processing may be performed. In the present exemplary embodiment, a desired analysis type can be specified by selecting an analysis type indicated by Density Analysis 903 or an item 905 displayed by selection of a Tools button 904 in FIG. 6A, and, if needed, an item 912 related to the number of analysis dimensions. The analysis processing according to the foregoing exemplary embodiment can thus be performed, for example, based on the operator's instructions, using a motion contrast image of which the image quality is enhanced by the trained model for image quality enhancement. This can improve the accuracy of the analysis processing according to the foregoing exemplary embodiment, for example.

Now, the execution of the image quality enhancement processing upon screen transition will be described with reference to FIGS. 6A and 6B. FIG. 6A illustrates an example of the report screen on which the OCTA image of FIG. 6B is displayed on an enlarged scale. In FIG. 6A, the button 911 is also displayed like FIG. 6B. The screen transition from FIG. 6B to FIG. 6A is effected by double-clicking the OCTA image, for example. The transition from FIG. 6A to FIG. 6B is effected by using a close button (not illustrated). Note that the screen transitions are not limited to the methods described here, and a non-illustrated user interface may be used.

If the image quality enhancement processing is specified to be executed (the button 911 is active) at the time of image transition, the state is maintained even after the screen transition. More specifically, if the screen of FIG. 6B is displaying a high image quality image when the screen transitions to that of FIG. 6A, the screen of FIG. 6A also displays a high image quality image. The button 911 is then kept activated. The same applies to the transition from FIG. 6A to FIG. 6B. In FIG. 6A, the display can be switched to a low image quality image by specifying the button 911.

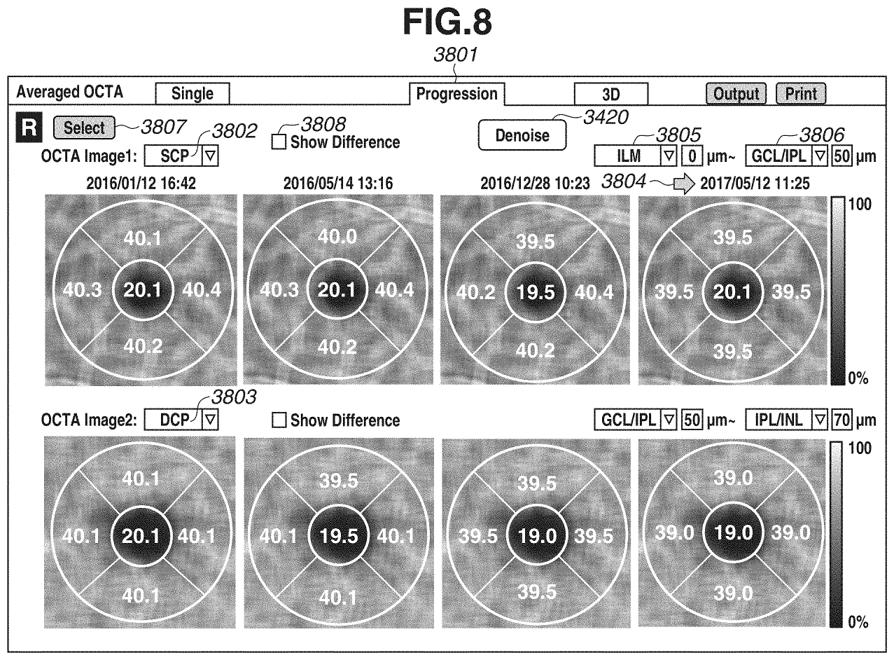

The screen transitions are not limited to the screen transitions described here, and the state of displaying a high image quality image is maintained as long as a transition occurs to a screen displaying the same imaging data, such as a display screen for follow-up and a panoramic display screen. In other words, an image corresponding to the state of the image quality enhancement button on the display screen before a transition is displayed on the display screen after the transition. For example, if the image quality enhancement button on the display screen before a transition is activated, a high image quality image is displayed on the display screen after the transition. For example, if the image quality enhancement button on the display screen before a transition is deactivated, a low image quality image is displayed on the display screen after the transition. If the image quality enhancement button on the display screen for follow-up (for example, a button 3420 in FIG. 8 to be described below) is activated, a plurality of images arranged and displayed on the display screen for follow-up, obtained at different dates and times (on different examination dates), may be switched to high image quality images. In other words, the information processing apparatus may be configured such that if the image quality enhancement button on the display screen for follow-up is activated, the activation is reflected on a plurality of images obtained at different dates and times in a collective manner.

FIG. 8 illustrates an example of the display screen for follow-up. If a tab 3801 is selected based on the examiner's instructions, the display screen for follow-up is displayed as illustrated in FIG. 8. Here, the examiner can change the depth range of the analysis target area by making selections from predetermined depth range sets displayed in list boxes (3802 and 3803). For example, the retinal surface layer is selected in the list box 3802, and the retinal deep layer is selected in the list box 3803. The analysis results of motion contrast images of the retinal surface layer are displayed in the upper display areas. The analysis results of motion contrast images of the retinal deep layer are displayed in the lower display areas. In other words, if a depth range is selected, a plurality of images of different dates and times is collectively switched to a juxtaposed display of the analysis results of a plurality of motion contrast images in the selected depth range.

Here, if the display of the analysis results is deselected, the display of the analysis results may be collectively switched to a juxtaposed display of a plurality of motion contrast images of different dates and times. If the button 3420 is then specified based on the examiner's instructions, the display of the plurality of motion contrast images is collectively switched to the display of a plurality of high image quality images. The button 3420 is an example of the image quality enhancement button, like the button 911 in FIGS. 6A and 6B described above.

If the display of the analysis results is selected and the button 3420 is specified based on the examiner's instructions, the display of the analysis results of the plurality of motion contrast images is collectively switched to the display of the analysis results of the plurality of high image quality images. Here, the analysis results may be displayed by being superimposed on the images with a given transparency. The switch to the display of the analysis results here may be effected by switching to a state where the analysis results are superimposed on the displayed images with a given transparency, for example. The switch to the display of the analysis results may effected by switching to display of images (for example, two-dimensional maps) obtained by performing blending processing on the analysis results and the images with a given transparency, for example.

A layer boundary type and an offset position to be used in specifying the depth ranges can be collectively changed from a user interface like list boxes 3805 and 3806. The depth range of a plurality of motion contrast images of different dates and times may be collectively changed by displaying a tomographic image as well and moving layer boundary data superimposed on the tomographic image based on the examiner's instructions. Here, a plurality of tomographic images of different dates and times may be displayed in a row, and if the foregoing movement is made on one of the tomographic images, the layer boundary data may be similarly moved on the other tomographic images. Moreover, the image projection method and the presence or absence of projection artifact suppression processing may be changed, for example, by making selections from a user interface such as a context menu. A selection button 3807 may be selected to display a selection screen, and images selected from an image list displayed on the selection screen may be displayed. An arrow 3804 display in the upper part of FIG. 8 is a symbol indicating the currently selected examination, with the examination selected at the time of follow-up imaging (the leftmost image in FIG. 8) as a reference examination (baseline). It will be understood that a symbol indicating the reference examination may be displayed on the display unit.

If a “Show Difference” check box 3808 is specified, an analysis value distribution (map, sector map) for a reference image is displayed on the reference image. In such a case, differential analysis value maps between the analysis value distribution calculated for the reference image and those calculated for the images displayed in areas corresponding to the other examination dates are displayed in the respective areas. A trend graph (graph of analysis values for the images of the respective examination dates, obtained by temporal change analysis) may be displayed as an analysis result on the report screen. In other words, time series data (for example, time series graph) on the plurality of analysis results corresponding to the plurality of images of different dates and times may be displayed. Here, the analysis results of dates and times different from the plurality of dates and times corresponding to the plurality of displayed images may also be displayed as time series data in a manner distinguishable from the plurality of analysis results corresponding to the plurality of displayed images (for example, the points on the time series graph are in different colors depending on the presence or absence of image display). A regression line (curve) and/or a corresponding equation of the trend graph may be displayed on the report screen.

The present exemplary embodiment has dealt with motion contrast images. However, this is not restrictive. The images to be subjected to the processing according to the present exemplary embodiment, such as display, image quality enhancement, and image analysis, may be tomographic images. The images are not limited to tomographic images, either, and other images such as SLO images, fundus photographs, and fluorescent fundus photographs may be used as well. In such a case, user interfaces for performing the image quality enhancement processing may include one for giving an instruction to perform the image quality enhancement processing on a plurality of different types of images, and one for giving an instruction to perform the image quality enhancement processing on an image or images selected from the plurality of different types of images.

For example, the target images of the image quality enhancement processing may be an OCTA front image corresponding to one depth range instead of a plurality of OCTA front images (OCTA en-face images, motion contrast en-face images) (corresponding to a plurality of depth ranges). The target images of the image quality enhancement processing may be luminance front images (luminance en-face images), B-scan OCT tomographic images, or B-scan tomographic images of motion contrast data (OCTA tomographic images), for example, instead of OCTA front images. The target images of the image quality enhancement processing may include not only OCTA front images, but also various medical images including luminance front images, B-scan OCT tomographic images, and B-scan tomographic images of motion contrast data (OCTA tomographic images), for example. In other words, the target images of the image quality enhancement processing may be at least one of various medical images displayed on the display screen of the display unit, for example. Here, trained models for image quality enhancement corresponding to the respective types of target images of the image quality enhancement processing may be used, for example, since the feature amounts of the images can vary from one image type to another. For example, the information processing apparatus may be configured such that if the button 911 or the button 3420 is pressed, the image quality enhancement processing is not only performed on OCTA front images using a trained model for image quality enhancement corresponding to OCTA front images but also performed on OCT tomographic images using a trained model for image quality enhancement corresponding to OCT tomographic images. For example, the information processing apparatus may be configured to, if the button 911 or the button 3420 is pressed, not only switch to display of high image quality OCTA front images generated using the trained model for image quality enhancement corresponding to OCTA front images but also switch to display of high image quality OCT tomographic images generated using the trained model for image quality enhancement corresponding to OCT tomographic images. Here, the information processing apparatus may be configured such that lines indicating the positions of the OCT tomographic images are superimposed on the OCTA front images. The lines may be configured to be movable on the OCTA front images based on the examiner's instructions. The information processing apparatus may also be configured to, if the display of the button 911 or the button 3420 is activated and the lines are moved, switch to display of high image quality OCT tomographic images obtained by performing the image quality enhancement processing on the OCT tomographic images corresponding to the current line positions. The information processing apparatus may be configured to display image quality enhancement buttons corresponding to the button 3420 for the respective target images of the image quality enhancement processing so that the image quality enhancement processing can be independently performed on each image.

Moreover, information indicating blood vessel regions in OCTA tomographic images (for example, motion contrast data higher than or equal to a threshold value) may be superimposed on OCT tomographic images that are the B-scans at the corresponding positions. For example, if the OCT tomographic images here are enhanced in image quality, the OCTA tomographic images at the corresponding positions may be enhanced in image quality. Information indicating the blood vessel regions in the OCTA tomographic images obtained by the image quality enhancement then may be displayed in a superimposed manner on the OCT tomographic images obtained by the image quality enhancement. The information indicating blood vessel regions may be any information identifiable in terms of color etc. The information processing apparatus may be configured such that the information indicating the blood vessel regions can be switched between being superimposed display and being hidden based on the examiner's instructions. If the line indicating the position of an OCT tomographic image is moved on an OCTA front image, the display of the OCT tomographic image may be updated based on the position of the line. Here, since the OCTA tomographic image at the corresponding position is also updated, the superimposed display of the information indicating blood vessel regions, obtained from the OCTA tomographic image, may be updated. This enables, for example, effective observation of a three-dimensional distribution and a state of the blood vessel regions at a given position while easily checking the positional relationship between the blood vessel regions and a region of interest. The image quality of an OCTA tomographic image may be enhanced by image quality enhancement processing such as addition average processing on a plurality of OCTA tomographic images obtained at the corresponding position instead of using the trained model for image quality enhancement. An OCT tomographic image may be a pseudo OCT tomographic image reconstructed as a cross section of the OCT volume data at a given position. An OCTA tomographic image may be a pseudo OCTA tomographic image reconstructed as a cross section of the OCTA volume data at a given position. The given position may be at least one arbitrary position, and the information processing apparatus may be configured such that the given position can be changed based on the examiner's instructions. Here, the information processing apparatus may be configured to reconstruct a plurality of pseudo tomographic images corresponding to a plurality of positions.

Only one or a plurality of tomographic graphic images (for example, an OCT tomographic image or images, or an OCTA tomographic image or images) may be displayed. If a plurality of tomographic images is displayed, tomographic images obtained at respective difference positions in a sub scanning direction may be displayed. If, for example, a plurality of tomographic images obtained by cross scanning or the like is displayed with enhanced image quality, images in respective different scanning directions may be displayed. If, for example, a plurality of tomographic images obtained by radial scanning or the like is displayed with enhanced image quality, selected some of the plurality of tomographic images (for example, two tomographic images at mutually symmetrical positions with respect to a reference line) may be displayed. Moreover, a plurality of tomographic images may be displayed on a display screen for follow-up such as illustrated in FIG. 8, and image quality enhancement instructions may be given and analysis results (such as the thickness of a specific layer) may be displayed by using techniques similar to the foregoing. The image quality enhancement processing may be performed on tomographic images based on information stored in a database by using techniques similar to the foregoing.

Similarly, in displaying an SLO fundus image with enhanced image quality, for example, SLO fundus images displayed on the same display screen may be displayed with enhanced image quality. In displaying a luminance front image with enhanced image quality, for example, luminance front images displayed on the same display screen may be displayed with enhanced image quality. Moreover, a plurality of SLO fundus images or luminance front images may be displayed on a display screen for follow-up such as illustrated in FIG. 8, and image quality enhancement instructions may be given and analysis results (such as the thickness of a specific layer) may be displayed by using techniques similar to the foregoing. The image quality enhancement processing may be performed on SLO fundus images and luminance front images based on information stored in a database by using techniques similar to the foregoing. Note that the display of the tomographic images, SLO fundus images, and luminance front images is illustrative, and such images may be displayed in any mode depending on a desired configuration. At least two or more of OCTA front images, tomographic images, SLO fundus images, and luminance front images may be enhanced in image quality and displayed based on a single instruction.

With such a configuration, the display control unit 103 can display images processed by the image quality enhancement unit (not illustrated) according to the present exemplary embodiment on the display unit. As described above, if at least one of a plurality of conditions related to the display of high image quality images, the display of analysis results, the depth range of front images to be displayed, and the like is selected, the selected state may be maintained even after transition of the display screen.

As described above, if at least one of the plurality of conditions is being selected, the state where the at least one condition is being selected may be maintained even after another condition is selected. For example, if the display of analysis results is being selected, the display control unit 103 may switch the display of the analysis results of low image quality images to the display of the analysis results of high image quality images based on the examiner's instructions (for example, when the button 911 or the button 3420 is specified). If the display of analysis results is being selected, the display control unit 103 may switch the display of the analysis results of high image quality images to the display of the analysis results of low image quality images based on the examiner's instructions (for example, when the button 911 or the button 2420 is unspecified).

If the display of high image quality images is being deselected, the display control unit 103 may switch the display of the analysis results of low image quality images to the display of the low image quality images based on the examiner's instructions (for example, when the display of analysis results is unspecified). If the display of high image quality images is being deselected, the display control unit 103 may switch the display of low image quality images to the display of the analysis results of the low image quality images based on the examiner's instructions (for example, when the display of analysis results is specified). If the display of high image quality images is being selected, the display control unit 103 may switch the display of the analysis results of high image quality images to the display of the high image quality images based on the examiner's instructions (for example, when the display of analysis results is unspecified). If the display of high image quality images is being selected, the display control unit 103 may switch the display of high image quality images to the display of the analysis results of the high image quality images based on the examiner's instructions (for example, when the display of analysis results is specified).

Suppose a case where the display of high image quality images is being deselected and the display of a first type of analysis result is being selected. In such a case, the display control unit 103 may switch the display of the first type of analysis result of a low image quality image to the display of a second type of analysis result of the low image quality image based on the examiner's instructions (for example, when the display of the second type of analysis result is specified). Now, suppose a case where the display of a high image quality image is being selected and the display of the first type of analysis result is being selected. In such a case, the display control unit 103 may switch the display of the first type of analysis result of a high image quality image to the display of the second type of analysis result of the high image quality image based on the examiner's instructions (for example, when the display of the second type of analysis result is specified).

The display screen for follow-up may be configured such that such changes in display are reflected on a plurality of images obtained at different dates and times in a collective manner as described above. Here, the analysis results may be displayed by being superimposed on the images with a given transparency. The switching to the display of the analysis results may be effected by switching to a state where the analysis results are superimposed on the displayed images with a given transparency, for example. The switching to the display of the analysis results may be effected by switching to display of images (for example, two-dimensional maps) obtained by blending the analysis results and the images with a given transparency, for example.

In the foregoing exemplary embodiment, the display control unit 103 can display on the display unit an image selected from among high image quality images generated by the image quality enhancement unit and input images based on the examiner's instructions. The display control unit 103 may also switch the display on the display screen of the display unit from a captured image (input image) to a high image quality image based on the examiner's instructions. In other words, the display control unit 103 may switch the display of a low image quality image to the display of a high image quality image based on the examiner's instructions. The display control unit 103 may also switch the display of a high image quality image to the display of a low image quality image based on the examiner's instructions.

Moreover, the image quality enhancement unit of the information processing apparatus may start the image quality enhancement processing using an image quality enhancement engine (trained model for image quality enhancement) (may input an image into the image quality enhancement engine) based on the examiner's instructions, and the display control unit 103 may display the high image quality image generated by the image quality enhancement unit on the display unit. Alternatively, the image quality enhancement engine may automatically generate a high image quality image based on an input image when the input image is captured by an imaging device (OCT), and the display control unit 103 may display the high image quality image on the display unit based on the examiner's instructions. Here, the image quality enhancement engine includes a trained model that performs the foregoing image quality improvement processing (image quality enhancement processing).

Such processes may also be similarly performed on the output of analysis results. More specifically, the display control unit 103 may switch the display of an analysis result of a low image quality image to the display of an analysis result of a high image quality image based on the examiner's instructions. The display control unit 103 may switch the display of an analysis result of a high image quality image to the display of an analysis result of a low image quality image based on the examiner's instructions. It will be understood that the display control unit 103 may switch the display of an analysis result of a low image quality image to the display of the low image quality image based on the examiner's instructions. The display control unit 103 may switch the display of a low image quality image to the display of an analysis result of the low image quality image based on the examiner's instructions. The display control unit 103 may switch the display of an analysis result of a high image quality image to the display of the high image quality image based on the examiner's instructions. The display control unit 103 may switch the display of a high image quality image to the display of an analysis result of the high image quality image based on the examiner's instructions.

The display control unit 103 may also switch the display of an analysis result of a low image quality image to the display of another type of analysis result of the low image quality image based on the examiner's instructions. The display control unit 103 may switch the display of an analysis result of a high image quality image to the display of another type of analysis result of the high image quality image based on the examiner's instructions.

Here, the analysis result of a high image quality image may be displayed by being superimposed on the high image quality image with a given transparency. The analysis result of a low image quality image may be displayed by being superimposed on the low image quality image with a given transparency. The switching to the display of an analysis result may be effected by switching to a state where the analysis result is superimposed on the displayed image with a given transparency, for example. The switching to the display of an analysis result may be effected by switching to display of an image (for example, two-dimensional map) obtained by blending the analysis result and the image with a given transparency, for example.

### FIRST MODIFICATION

The information processing apparatus according to the foregoing exemplary embodiment has an automatic transfer function of automatically transferring imaging data captured by the ophthalmologic imaging device or data converted into a specified format to a storage location specified in advance. Possible use cases of the automatic transfer function in actual hospital operations includes a case where the data is transferred to a recording and archiving system (so-called picture archiving and communication system (PACS)) and a case where the data is transferred to an electronic medical record and other diagnostic systems. In such operations, different data contents have sometimes been demanded to be transferred depending on the ophthalmologic imaging devices. For example, in transferring imaging data to the recording and archiving system, fundus images are often transferred from a fundus camera, and tomographic images from an OCT. In transferring data to a diagnostic system, fundus images are often transmitted from a fundus camera, and report images including analysis results such as a retinal thickness map from an OCT instead of tomographic images. The information processing apparatus according to the foregoing exemplary embodiment can deal with even such use cases, for example.

In the foregoing exemplary embodiments, the information processing apparatus may be configured such that the examiner can manually transmit (manually transfer) individual pieces of imaging data from a display screen such as a report screen. For example, the information processing apparatus may be configured such that if a manual transmission button is pressed on the report screen based on the examiner's instructions, images displayed on the report screen or a report image corresponding to the report screen is transmitted as imaging data. For example, if an examination is specified on a patient screen based on the examiner's instructions, imaging data related to the specified examination may be made transmittable based on the examiner's instructions. If a patient is specified on the patient screen based on the examiner's instructions, imaging data related to at least one examination on the specified patient may be made transmittable based on the examiner's instructions.

The information processing apparatus may be configured such that if a button for giving an instruction to display high image quality images (image quality enhancement button) is set to be activated (image quality enhancement processing to be on) by default on an initial display screen of the report screen, a report image corresponding to a report screen including high image quality images and the like is transmitted to a server based on the examiner's instructions. The information processing apparatus may be configured such that if the button for giving an instruction to display high image quality images is set to be activated by default, the report image corresponding to the report screen including high image quality images and the like is (automatically) transmitted to the server at the end of examination (for example, when an imaging confirmation screen or a preview screen is switched to the preview screen based on the examiner's instructions). Here, the information processing apparatus may be configured such that a report image generated based on various settings included in default settings (for example, settings related to at least one of the following: the depth range for generating an en-face image on the initial display screen of the report screen, the presence or absence of superimposition of an analysis map, whether an image is a high image quality image, and whether the display screen is for follow-up) is transmitted to the server.

### SECOND MODIFICATION

In the foregoing exemplary embodiments, the types of transmittable images in the transmission settings may include not only at least one tomographic image (B-scan image) but a front image or images (en-face image(s)). Here, data including a high image quality image (second medical image) obtained from a low image quality image (first medical image) corresponding to imaging data by using the trained model for image quality enhancement (image quality enhancement model, image quality enhancement engine) and the low image quality image as a set may be transmittable. Here, the data may be transmittable to a training data server for additional training. Managing data in the form of the foregoing set, even on a server not intended for such a purpose, facilitates the use of the set as training data in generating the trained model for image quality enhancement. The trained model for image quality enhancement may be a trained model (machine learning model, machine learning engine) obtained by performing machine learning with training data including low image quality images as input data and high image quality images as ground truth data (teaching data).

The foregoing trained model can be obtained by machine learning using training data. Examples of the machine learning include deep learning with a multilevel neural network. For example, a convolutional neural network (CNN) can be used as a machine learning model for at least part of the multilevel neural network. A technique related to an auto-encoder may be used for at least part of the multilevel neural network. A technique related to backpropagation may be used for training. Note that the machine learning is not limited to deep learning, and any learning using a model that can extract (express) feature amounts of training data such as images by itself through training may be employed. A trained model refers to a machine learning model based on a given machine learning algorithm, trained with appropriate training data in advance. Note that a trained model shall refer to one capable of additional training, not one not to be trained further. Training data includes pairs of input data and output data (ground truth data). As employed herein, training data may be referred to as teaching data, and ground truth data may be referred to as teaching data. The image quality enhancement engine may be a trained model obtained by additional training with training data including at least one high image quality image generated by the image quality enhancement engine. Here, the information processing apparatus may be configured such that whether to use the high image quality image as training data for additional training can be selected based on the examiner's instructions. (Third Modification)

The display control unit 103 in the foregoing various exemplary embodiments and modifications may display analysis results such as the thickness of a desired layer and various blood vessel densities on the report screen that is a display screen. The display control unit 103 may also display, as analysis results, parameter values (distribution) related to a region of interest including at least one of the following: an optic disc, macula, blood vessel region, nerve fiber bundle, vitreous region, macular region, choroidal region, scleral region, sieve plate region, retinal layer boundary, retinal layer boundary end, visual cells, blood cells, blood vessel wall, vessel inner wall boundary, vessel outer wall boundary, ganglion cells, corneal region, corner region, and Schlemm's canal. Here, for example, an accurate analysis result can be displayed by analyzing a medical image to which various types of artifact reduction processing are applied. Examples of artifacts may include a false image area occurring due to light absorption in a blood vessel region and the like, a projection artifact, and a band-like artifact occurring in the main scanning direction of measurement light in a front image due to the state (such as a movement or a blink) of the eye to be examined An artifact may refer to any imaging error area occurring in a medical image of a predetermined region of the examinee at random in each imaging operation. Parameter values (distribution) related to an area including at least one of the foregoing various artifacts (imaging error areas) may also be displayed as an analysis result. Parameter values (distribution) related to an area including at least one of abnormal regions such as a drusen, newborn blood vessel, vitiligo (hard vitiligo), and pseudodrusen may be displayed as an analysis result. A comparison result obtained by comparing a standard value or standard range obtained using a standard database with an analysis result may be displayed.

Analysis results may be displayed as an analysis map, sectors indicating corresponding statistics in the respective sections, and the like. Analysis results may be ones generated by using a trained model (analysis result generation engine, trained model for analysis result generation) obtained by training with analysis results of medical images as training data. Here, the trained model may be one obtained by training using training data including medical images and analysis results of the medical images, training data including medical images and analysis results of a different type of medical images from the medical images, or the like.

The trained model may be one obtained by training using training data including input data with a plurality of different types of medical images of a predetermined portion as a set, like a luminance front image (luminance tomographic image) and a motion contrast front image. Here, a luminance front image corresponds to a tomographic en-face image, and a motion contrast front image corresponds to an OCTA en-face image.

The information processing apparatus may be configured such that analysis results obtained by using high image quality images generated by the image quality enhancement engine are displayed. The trained model for image quality enhancement may be one obtained by training with training data including a first image as input data and a second image having higher image quality than the first image as ground truth data. For example, the second image may be a high image quality image that is enhanced in contrast and reduced in noise by superposition processing of a plurality of first images (for example, averaging processing of a plurality of aligned first images), etc.

The input data included in the training data may include high image quality images generated by the image quality enhancement engine, or a set of low and high image quality images.

For example, the training data may be data including labeled (annotated) input data, with information including at least one of the following as ground truth data (for supervised learning): analytic values (such as an average and a median) obtained by analyzing analysis areas, a table containing analytic values, an analysis map, and the positions of analysis areas such as sectors in an image. The information processing apparatus may be configured such that analysis results obtained by the trained model for analysis result generation are displayed based on the examiner's instructions.

The display control unit 103 in the foregoing exemplary embodiments and modifications may display various diagnostic results, such as those of glaucoma and age-related macular degeneration, on the report screen that is a display screen. Here, an accurate diagnostic result can be displayed by analyzing medical images to which the foregoing various types of artifact reduction processing are applied, for example. The position of an identified abnormal region may be displayed on the image as a diagnostic result. The state of the abnormal region and the like may be displayed using characters and the like. A classification result (such as Curtin's classification) of abnormal regions and the like may be displayed as a diagnostic result. Information indicating the likelihood of respective abnormal regions (for example, numerical values indicating rates) may be displayed as the classification result. Information needed for a doctor to make a diagnosis may be displayed as a diagnostic result. Examples of the needed information include advice for additional imaging and the like. For example, if an abnormal region is detected in a blood vessel region in an OCTA image, a message to do additional fluorescence imaging using a contrast agent, capable of more detailed observation of blood vessels than with OCTA, may be displayed. A diagnostic result may be information about the future diagnostic plan for the examinee A diagnostic result may be information including, for example, at least one of the following: a diagnostic name, the type and state (degree) of a lesion (abnormal region), the position of the lesion in an image, the position of the lesion relative to a region of interest, observations (radiogram interpretation observations and the like), grounds for the diagnostic name (such as positive medical support information), and grounds against the diagnostic name (negative medical support information). Here, a diagnostic result more probable than with a diagnostic name input based on the examiner's instructions may be displayed as medical support information, for example. If a plurality of types of medical images is used, a type or types of medical images that can be the grounds for the diagnostic result may be displayed in an identifiable manner.

The diagnostic results may be ones generated by using a trained model (diagnostic result generation engine, trained model for diagnostic result generation) trained with diagnostic results of medical images as training data. The trained model may be one obtained by training using training data including medical images and the diagnostic results of the medical images, training data including medical images and the diagnostic results of a different type of medical images from the medical images, or the like. The information processing apparatus may be configured to display a diagnostic result obtained by using a high image quality image generated by the image quality enhancement engine.

The input data included in the training data may be high image quality images generated by the image quality enhancement engine, or a set of low and high image quality images. The training data may be data including labeled (annotated) input data, with information including at least one of the following as ground truth data (for supervised learning): a diagnostic name, the type and state (degree) of a lesion (abnormal region), the position of the lesion in an image, the position of the lesion relative to a region of interest, observations (radiogram interpretation observations and the like), grounds for the diagnostic name (such as positive medical support information), and grounds against the diagnostic name (such as negative medical support information). The information processing apparatus may be configured such that a diagnostic result obtained by the trained model for diagnostic result generation is displayed based on the examiner's instructions.

The foregoing various trained models may be trained not only by supervised learning (training with labeled training data) but by semi-supervised learning. Semi-supervised learning is a technique that includes, for example, training each of a plurality of discriminators (classifiers) by supervised learning, and then discriminating (classifying) unlabeled training data, automatically labeling (annotating) the discrimination results (classification results) based on their reliabilities (for example, labeling discrimination results having a likelihood higher than or equal to a threshold), and performing training with the labeled training data. An example of semi-supervised learning may be co-training (multiview). For example, the trained model for diagnostic result generation may be a trained model obtained by performing semi-supervised learning (for example, co-training) using a first discriminator for discriminating a medical image of a normal test subject and a second discriminator for discriminating a medical image including a specific lesion. The trained model is not limited to diagnostic purposes and may be directed to imaging assistance and the like, for example. In such a case, the second discriminator may be one for discriminating a medical image including a partial area, such as a region of interest and an artifact area.

The display control unit 103 in the foregoing various exemplary embodiments and modifications may display an object recognition result (object detection result) or a segmentation result of a partial area, such as a region of interest, an artifact area, and an abnormal region mentioned above, on the report screen that is a display screen. Here, for example, a rectangular frame or the like may be displayed in a superimposed manner around the object on the image. For example, color or the like may be displayed in a superimposed manner on the object in the image. The object recognition result or segmentation result may be one generated using a trained model (object recognition engine, trained model for object recognition, segmentation engine, or trained model for segmentation) obtained by training using training data including medical images labeled (annotated) with information indicating object recognition or segmentation as ground truth data. The foregoing analysis result or diagnostic result may be obtained by using the foregoing object recognition result or segmentation result. For example, the processing for generating an analysis result or diagnostic result may be performed on a region of interest obtained by the object recognition or segmentation processing.

To detect an abnormal region, the information processing apparatus may use a generative adversarial networks (GAN) or a variational auto-encoder (VAE). For example, a deep convolutional GAN (DCGAN) including a generator trained to generate a tomographic image and a discriminator trained to discriminate a new tomographic image generated by the generator from an actual tomographic image can be used as a machine learning model.

In the case of using a DCGAN, for example, the discriminator encodes an input tomographic image into latent variables, and the generator generates a new tomographic image based on the latent variables. A difference between the input tomographic image and the generated new tomographic image then can be extracted (detected) as an abnormal region. In the case of using a VAE, for example, an encoder encodes an input tomographic image into latent variables, and a decoder decodes the latent variables to generate a new tomographic image. A difference between the input tomographic image and the generated new tomographic image then can be extracted as an abnormal region. While a tomographic image is described as an example of input data, a fundus image, an anterior eye front image, or the like may be used.

The information processing apparatus may further use a convolutional auto-encoder (CAE) to detect an abnormal region. In the case of using a CAE, the CAE is trained with the same images, as input data and output data. Consequently, if an image including an abnormal region is input into the CAE for estimation, an image including no abnormal region is output based on the tendency of the training. The difference between the image input to the CAE and the image output from the CAE then can be extracted as an abnormal region. Even in such a case, not only a tomographic image but a fundus image, an anterior eye front image, and the like may be used as input data.

In such cases, the information processing apparatus can generate information about a difference between a medical image obtained by using a generative adversarial network or an auto-encoder and a medical image input to the generative adversarial network or the auto-encoder as information about an abnormal region. The information processing apparatus can thus be expected to accurately detect an abnormal region at high speed. For example, even if a large number of medical images including abnormal regions are difficult to collect as training data for improved detection accuracy of abnormal regions, medical images of normal test subjects, which are relatively easy to collect in numbers, can be used as training data. This enables, for example, efficient training for detecting an abnormal region with high accuracy. As employed herein, auto-encoders include a VAE and a CAE. At least part of the generation unit in a generative adversarial network may be constituted by a VAE. This enables, for example, generation of relatively sharp images while reducing a phenomenon where similar pieces of data are generated. For example, the information processing apparatus can generate information about differences between medical images generated from various medical images by using a generative adversarial network or auto-encoder and the medical images input to the generative adversarial network or auto-encoder as information about abnormal regions. For example, the display control unit 103 can display the information about the differences between the medical images generated from various medical images by using the generative adversarial network or auto-encoder and the medical images input to the generative adversarial network or auto-encoder on the display unit as information about abnormal regions.

A diseased eyes has different image features depending on the type of disease. The trained models used in the foregoing various exemplary embodiments and modifications may therefore be generated and provided for each type of disease or each abnormal region. In such a case, for example, the information processing apparatus can select a trained model or models to be used for processing based on the operator's input (instructions) about the type of disease, the abnormal region, and the like of the eye to be examined Note that the trained models prepared for each type of disease or each abnormal region are not limited to ones used to detect retinal layers and generate an area-labeled image, etc. For example, trained models to be used as an image evaluation engine, an analysis engine, and the like may be prepared. Here, the information processing apparatus may discriminate the type of disease and the abnormal region of the eye to be examined from an image by using separately-prepared trained models. In such a case, the information processing apparatus can automatically select the trained models to be used for the foregoing processing based on the type of disease and the abnormal region discriminated using the separately-prepared trained models. The trained models for discriminating the type of disease and the abnormal region of the eye to be examined may be trained with pairs of training data with tomographic images, fundus images, and the like as input data, and the types of disease and abnormal regions in such images as output data. Tomographic images, fundus images, and the like may be used by themselves as the input data in the training data. A combination of these may be used as the input data.

The trained model for diagnostic result generation in particular may be a trained model obtained by training using training data including input data with a plurality of different types of medical images of a predetermined region of an examinee as a set. Examples of the input data include in the training data here may include input data with a motion contrast front image and a luminance front image (or luminance tomographic image) of the fundus as a set, and input data with a tomographic image (B-scan image) and a color fundus image (or fluorescent fundus image) of the fundus as a set. Any plurality of different types of medical images obtained by different modalities, different optical systems, different principles, and the like may be used.

The trained model for diagnostic result generation in particular may be a trained model obtained by training using training data including input data with a plurality of medical images of different regions of an examinee as a set. Examples of the input data included in the training data here may include input data with a tomographic image (B-scan image) of the fundus and a tomographic image (B-scan image) of the anterior eye part as a set, and input data with a three-dimensional OCT image of the macula in the fundus and a circle scan (or raster scan) tomographic image of the optic disc in the fundus as a set.

The input data included in the training data may be a plurality of different types of medical images of different regions of an examinee An example of the input data included in the training data here may be input data including a tomographic image of the anterior eye part and a color fundus image as a set. Moreover, the foregoing various trained models may be ones obtained by training using training data including input data with a plurality of medical images of a predetermined region of an examinee at different imaging angles of view as a set. The input data included in the training data may be a connected plurality of medical images obtained by dividing a predetermined region into a plurality of areas in a time divisional manner like a panoramic image. Here, accurate image feature amounts can be obtainable since the use of a wide-angle image like a panoramic image as the training data provides a greater amount of information than with a narrow-angle image. The results of respective processes can thus be improved. The input data included in the training data may be input data including a plurality of medical images of a predetermined region of an examinee as of different dates and times as a set.

The display screen displaying at least one of the foregoing analysis result, diagnostic result, object recognition result, and segmentation result is not limited to a report screen. For example, such a display screen may be displayed as at least one of the following: an imaging confirmation screen, a display screen for follow-up, and preview screens for various adjustments before imaging (display screens displaying various types of live moving images). For example, the at least one of the results obtained by using the foregoing various trained models may be displayed on the imaging confirmation screen, whereby the examiner can observe an accurate result even immediately after imaging. For example, the information processing apparatus may be configured such that if a specific object is recognized, a frame surrounding the recognized object is superimposed on a live moving image. Here, if information indicating the likelihood of the object recognition result (for example, a numerical value indicating a rate) exceeds a threshold, the frame surrounding the object may be highlighted, for example, in a different color or the like. The examiner can thus easily identify the object on the live moving image. The foregoing switching of display between a low image quality image and a high image quality image may be that between an analysis result of the low image quality image and an analysis result of the high image quality image.

The foregoing various trained models can be obtained by machine learning using training data. An example of the machine learning is deep leaning using a multilevel neural network. For example, a convolutional neural network (CNN) can be used as a machine learning model in at least part of the multilayer neural network. A technique related to an auto-encoder may be used for at least part of the multilayer neural network. A technique related to backpropagation may be used for training A technique (dropout) for deactivating units (neurons or nodes) at random may be used for training. A technique (batch normalization) for normalizing data delivered to each layer of the multilayer neural network before application of an activation function (for example, a rectified linear unit (ReLU) function) may be used for training. Note that the machine learning is not limited to deep learning, and any learning using a model that can extract (express) feature amounts of training data such as images by itself through training may be used. As employed herein, a machine learning model refers to a learning model using a machine learning algorithm such as deep learning. A trained model refers to a machine learning model using a given machine learning algorithm, trained with appropriate training data in advance. Note that a trained model refers to one capable of additional training, not one not to be trained further. Training data includes pairs of input data and output data (ground truth data). As employed herein, training data may be referred to as teaching data, and ground truth data may be referred to training data.

A GPU can efficiently perform calculations by processing more pieces of data in parallel. Performing processing using a GPU is thus effective in training a learning model a plurality of times, like deep learning. In the present modification, the processing by the information processing apparatus that is an example of a training unit (not illustrated) uses a GPU in addition to a CPU. Specifically, in executing a training program including a learning model, the CPU and the GPU perform training by executing computing in a cooperative manner. The computing in the processing of the training unit may be performed by either the CPU or the GPU alone. Like the training unit, a processing unit (estimation unit) that performs processing using the foregoing various trained models may also use a GPU. Moreover, the training unit may include a non-illustrated error detection unit and update unit. The error detection unit obtains an error between output data output from the output layer of the neural network in response to input data input to the input layer and ground truth data. The error detection unit may calculate the error between the output data from the neural network and the ground truth data by using a loss function. Based on the error obtained by the error detection unit, the update unit updates coupling weight coefficients and the like between nodes of the neural network to reduce the error. The update unit updates the coupling weight coefficients and the like by backpropagation, for example. Backpropagation is a technique for adjusting the coupling weight coefficients and the like between each node of the neural network so that the foregoing error decreases.

A U-net machine learning model having an encoder function with a plurality of levels of layers including a plurality of downsampling layers and a decoder function with a plurality of levels of layers including a plurality of upsampling layers can be applied to the machine learning models used for image quality enhancement, segmentation, and the like. A U-net machine learning model is configured such that position information (spatial information) made ambiguous in the plurality of levels of layers constituted as the encoder can be used at the levels of the same orders (mutually corresponding levels) in the plurality of levels of layers constituted as the decoder (for example, by using skip connections).

For example, a fully convolutional network (FCN), SegNet, and the like can be used as the machine learning models used for image quality enhancement, segmentation, and the like. A machine learning model for performing object recognition in units of regions based on a desired configuration may be used. For example, a region CNN (RCNN), a fast RCNN, or a faster RCNN can be used as the machine learning model for performing object recognition. Moreover, a You Only Look Once (YOLO) or a single shot detector or single shot multibox detector (SSD) can also be used as the machine learning model for performing object recognition in units of regions.

Examples of the machine learning models may include a capsule network (CapsNet). In a typical neural network, each unit (each neuron or each node) is configured to output a scalar value so that spatial information about a spatial positional relationship (relative position) between image features decreases, for example. This enables, for example, training such that the effects of local distortions, translations, and the like in the image decrease. By contrast, in a capsule network, each unit (each capsule) is configured to output spatial information as a vector so that spatial information is maintained, for example. This enables training taking into account a spatial positional relationship between image features, for example.

The image quality enhancement engine (trained model for image quality enhancement) may be a trained model obtained by additional training with training data including at least one high image quality image generated by the image quality enhancement engine. Here, the information processing apparatus may be configured such that whether to use the high image quality image as the training data for additional training can be selected based on the examiner's instructions. Such configurations are not limited to the trained model for image quality enhancement, and can be applied to the foregoing various trained models. The ground truth data used in training the foregoing various trained models may be generated by using a trained model for ground truth data generation for generating ground truth data such as labeled (annotated) data. Here, the trained model for ground truth data generation may be one obtained by (sequential) additional training using ground truth data labeled (annotated) by the examiner. More specifically, the trained model for ground truth data generation may be one obtained by additional training using training data with unlabeled data as input data and labeled data as output data. The information processing apparatus may be configured to correct the result of object recognition, segmentation, and the like in a frame determined to have low result accuracy among a plurality of consecutive frames such as those of a moving image, taking into account the results in previous and subsequent frames. Here, the information processing apparatus may be configured to perform additional training based on the examiner's instructions, with the corrected result as ground truth data.

In the foregoing various exemplary embodiments and modifications, in detecting partial areas (such as a region of interest, an artifact area, and an abnormal region) of the eye to be examined by using the trained model for object recognition or the trained model for segmentation, predetermined image processing can be applied to each detected area. Suppose, for example, a case where at least two of partial areas including a vitreous region, a retinal region, and a choroidal region are detected. In such a case, adjustments suitable for the respective areas can be made by using respective different image processing parameters in applying the imaging processing, such as contrast adjustment, to the at least two detected areas. Displaying the image where the suitable adjustments are made to the respective areas enables the operator to make a more appropriate diagnosis of disease and the like area by area. Note that the configuration using different image processing parameters for respective detected areas may also be similarly applied to areas of the eye to be examined that are detected without using a trained model.

### FOURTH MODIFICATION

The information processing apparatus may be configured such that the foregoing various trained models are used at least for each frame of a live moving image in the preview screen in the foregoing various exemplary embodiments and modifications. Here, the information processing apparatus may be configured such that if the preview screen displays a plurality of live moving images of different regions or a plurality of different types of live moving images, respective corresponding trained models are used for the live moving images. This, for example, can reduce the processing time of even the live moving images, and the examiner can obtain accurate information before the start of imaging. Diagnostic accuracy and efficiency can thus be improved since re-imaging failures and the like can be reduced, for example.

Examples of the plurality of live moving images may include a moving image of the anterior eye part for alignment in X, Y, and Z directions and a fundus front moving image for focus adjustment to the fundus observation optical system or for OCT focus adjustment. For example, the plurality of live moving images may be fundus tomographic moving images and the like for OCT coherence gate adjustment (to adjust a difference in optical path length between a measurement optical path length and a reference optical path length). Here, the information processing apparatus may be configured to make the foregoing various adjustments so that the areas detected using the foregoing trained model for object recognition or trained model for segmentation satisfy a predetermined condition. For example, the information processing apparatus may be configured to make various adjustments, such as an OCT focus adjustment, so that a value (such as a contrast value and an intensity value) related to a vitreous region or a predetermined retinal layer such as retinal pigment epithelium (RPE), detected using the trained model for object recognition or the trained model for segmentation, exceeds a threshold (or peaks). For example, the information processing apparatus may be configured to make an OCT coherence gate adjustment so that a vitreous region or a predetermined retinal layer such as RPE, detected using the trained model for object recognition or the trained model for segmentation, comes to a predetermined position in the depth direction.

In such cases, the image quality enhancement unit (not illustrated) of the information processing apparatus can generate a high image quality moving image by performing image quality enhancement processing on the moving image using a trained model. In addition, the control unit (not illustrated) of the information processing apparatus can control driving of an optical member for changing the imaging range, such as an OCT reference mirror (not illustrated), so that one of different regions identified by the segmentation processing or the like comes to a predetermined position in the display area where the high image quality moving image is displayed. In such a case, the control unit can automatically perform alignment processing so that the desired area comes to the predetermined position in the display area based on highly accurate information. For example, the optical member for changing the imaging range may be an optical member for adjusting a coherence gate position, and more specifically, may be a reference mirror or the like. The coherence gate position can be adjusted by an optical member that changes a difference in optical path length between the measurement optical path length and the reference optical path length. Examples of such an optical member may include a non-illustrated mirror for changing the optical path length of measurement light. The optical member for changing the imaging range may be a stage unit (not illustrated) of the imaging device, for example. The control unit may control driving of the scanning unit for scanning of measurement light so that the partial areas such as an artifact area obtained by the segmentation processing and the like are captured again (rescanned) during imaging or at the end of imaging, based on instructions related to the start of imaging. Moreover, the information processing apparatus may be configured, for example, to automatically make various adjustments and start imaging and the like if information (for example, a numerical value indicating a rate) indicating the likelihood of an object recognition result related to a region of interest exceeds a threshold. The information processing apparatus may be configured, for example, to switch to a state where various adjustments can be made and imaging can be started based on the examiner's instructions (cancel an execution prohibited state) if the information (for example, a numerical value indicating a rate) indicating the likelihood of the object recognition result related to the region of interest exceeds a threshold.

Moving images to which the foregoing various trained models can be applied are not limited to live moving images, and may be moving images stored (saved) in a storage unit, for example. Here, for example, a moving image obtained by performing alignment at least for each frame of a fundus tomographic moving image stored (saved) in a storage unit may be displayed on the display screen. For example, to observe the vitreous body in a suitable manner, a reference frame may initially be selected with reference to such a condition that the frame covers the vitreous body as much as possible. Here, each frame is a tomographic image (B-scan image) in X and Z directions. A moving image obtained by aligning the other frames to the selected reference frame in the X and Z directions then may be displayed on the display screen. Here, the information processing apparatus may be configured, for example, to successively display high image quality images (high image quality frames) sequentially generated from at least each frame of the moving image by the image quality enhancement engine.

As a technique for the foregoing frame-to-frame alignment, the same technique may be applied to or respective different techniques may be applied to the alignment in the X direction and the alignment in the Z direction (depth direction). Alignment in the same direction may be performed a plurality of times by using different techniques. For example, precise alignment may be performed after rough alignment. Examples of the alignment techniques include (rough Z-direction) alignment using a retinal layer boundary obtained by segmentation processing on the tomographic image (B-scan image), (precise X- and Z-direction) alignment using correlation information (similarity) between a plurality of areas obtained by dividing the tomographic image and the reference image, (X-direction) alignment using a one-dimensional projection image generated for each tomographic image (B-scan image), and (X-direction) alignment using a two-dimensional front image. The information processing apparatus may be configured to perform rough alignment in units of pixels and then perform precise alignment in units of subpixels.

During various adjustments, the imaging target such as the retina of the eye to be examined may not yet successfully imaged. An accurate high image quality image can therefore be not obtainable because of a large difference between the medical image input to the trained model and the medical images used as the training data. The information processing apparatus may therefore be configured to automatically start displaying a high image quality moving image (displaying high image quality frames in succession) if an image quality evaluation or other evaluation value for the tomographic image (B-scan) exceeds a threshold. The information processing apparatus may be configured to make an image quality enhancement button specifiable (activatable) by the examiner if the image quality evaluation or other evaluation value for the tomographic image (B-scan) exceeds the threshold. The image quality enhancement button is a button for specifying execution of the image quality enhancement processing. It will be understood that the image quality enhancement button may be a button for giving an instruction to display a high image quality image.

The information processing apparatus may be configured such that different trained models for image quality enhancement are prepared for respective imaging modes with different scanning patterns, etc., and the trained model for image quality enhancement corresponding to a selected imaging mode is selected. A single trained model for image quality enhancement obtained by training with training data including various medical images obtained in different imaging modes may be used.

### FIFTH MODIFICATION

In the foregoing various exemplary embodiments and modifications, if a trained model is under additional training, it can be difficult to make an output (estimation, prediction) by using the trained model being additionally trained itself. The information processing apparatus therefore is preferably configured to disable input of medical images other than training data into the trained model under additional training. Moreover, another trained model identical to the trained model before the execution of the additional training may be prepared as a backup trained model. Here, the information processing apparatus can be configured such that medical images other than training data can be input into the backup trained model while the additional training is in process. After the completion of the additional training, the additionally-trained trained model may be evaluated, and if no problem is found, the backup trained model may be replaced with the additionally-trained trained model. If any problem is found, the backup trained model may be used. The additionally-trained trained model may be evaluated, for example, by using a trained model for classification for classifying high image quality images obtained by the trained model for image quality enhancement from other types of images. For example, the trained model for classification may be a trained model obtained by training with training data that includes a plurality of images including high image quality images obtained by the trained model for image quality enhancement and low image quality images as input data and data labeled (annotated) with the types of images as ground truth data. Here, the estimated (predicted) image type of input data may be displayed along with information (such as numerical values indicating rates) indicating the likelihood of being respective image types included in the ground truth data during training. Aside from the foregoing images, the input data of the trained model for classification may include, in addition to the forgoing images, high image quality images that are enhanced in contrast and reduced in noise by superposition processing of a plurality of low image quality images (for example, averaging processing of a plurality of aligned low image quality images), etc. The additionally-trained trained model may be evaluated, for example, by comparing a plurality of high image quality images obtained from the same image using the additionally-trained trained model and the trained model yet not to be additionally trained (backup trained model), or comparing analysis results of the plurality of high image quality images. Here, for example, whether a comparison result of the plurality of high image quality images (an example of a change due to additional training) or a comparison result of the analysis results of the plurality of high image quality images (an example of a change due to additional training) falls within a predetermined range may be determined and the determination result may be displayed.

Trained models obtained by performing training for respective imaging regions may be made selectively usable. Specifically, the information processing apparatus may include a selection unit for selecting one of a plurality of trained models including a first trained model obtained by training with training data including a first imaging region (such as lungs or an eye to be examined) and a second trained model obtained by training with training data including a second imaging region different from the first imaging region. Here, the information processing apparatus may include a control unit (not illustrated) for performing additional training on the selected trained model. The control unit can search for data including the imaging region corresponding to the selected trained model and a captured image of the imaging region as a pair, and additionally train the selected trained model with the searched data as trained data based on the examiner's instructions. The imaging region corresponding to the selected trained model may be obtained from header information about the data or manually entered by the examiner. A server or the like in the hospital or an external institution such as a laboratory may be searched for the data via a network, for example. Additional training for each imaging region can thereby be efficiently performed by using captured images of the imaging region corresponding to the trained model.

The selection unit and the control unit may be constituted by software modules executed by a processor of the information processing apparatus, such as a CPU or a micro processing unit (MPU). The selection unit and the control unit may be constituted by circuits for providing specific functions, such as an application specific integrated circuit (ASIC), independent devices, or the like.

If training data for additional training is obtained from a server or the like in the hospital or an external institution such as a laboratory via a network, it is desirable to reduce a drop in reliability due to tampering, system troubles during the additional training, and the like. For that purpose, the validity of the training data for additional training may be detected by consistency check using a digital signature or hashing. This can protect the training data for additional training. If the validity of the training data for additional training is not successfully detected as a result of the consistency check using a digital signature or hashing, the information processing apparatus gives a warning and does not perform additional training with the training data. The installation location of the server is not limited. The server may have any configuration, such as a cloud server, a fog server, or an edge server.

The foregoing protection of data through consistency check is not limited to the training data for additional training, and can be applied to data including medical images. An image management system may be configured such that transactions of data including medical images between servers in a plurality of institutions are managed by a distributed network. The image management system may be configured such that a plurality of blocks each recording a transaction history with a hash value of the previous block is connected in a time series. Cryptography difficult to compute even using a quantum gate or other quantum computer (such as lattice-based cryptography and quantum key delivery-based quantum cryptography) may be used as a technique for performing consistency check and the like. Here, the image management system may be an apparatus and a system for receiving and storing images captured by imaging devices and image-processed images. The image management system can also transmit images based on a request from a connected device, perform image processing on the stored images, and request image processing from another device. Examples of the image management system may include a picture archiving and communication system (PACS). In particular, an image management system includes a database that can store received images along with various types of information including associated patient information and imaging times. The image management system is connected to a network, and can transmit and receive images, convert images, and transmit and receive various types of information associated with the stored images based on requests from other devices.

### SIXTH MODIFICATION

In the foregoing various exemplary embodiments and modifications, the examiner's instructions may be ones given by voice and the like aside from manual instructions (for example, instructions given from a user interface and the like). Here, for example, a machine learning engine (machine learning model) including a voice recognition engine (voice recognition model, trained model for voice recognition) obtained by machine learning may be used. The manual instruction may be an instruction given by character input or the like using a keyboard, a touch panel, or the like. Here, for example, a machine learning engine including a character recognition engine (character recognition model, trained model for character recognition) obtained by machine learning may be used. The examiner's instructions may also be instructions using gestures. Here, a machine learning engine including a gesture recognition engine (gesture recognition model, trained model for gesture recognition) obtained by machine learning may be used.

The examiner's instructions may be given as a detection result of the examiner's line of sight on the display screen (monitor) of the display unit. For example, the detection result of the line of sight may be a pupil detection result using the moving image of the examiner, captured from near the display screen (monitor) of the display unit. Here, the pupils in the moving image may be detected by using the foregoing object recognition engine. Furthermore, the examiner's instructions may be ones based on brain waves, a weak electrical signal flowing through the body, and the like.

In such cases, for example, the training data may include character data, voice data (waveform data), or the like indicating instructions to display the results of processing by the foregoing various trained models as input data and execution commands for actually displaying the results and the like of the processing by the various trained models on the display unit as ground truth data. For example, the training data may include character data, voice data, or the like indicating instructions to display a high image quality image obtained by the trained model for image quality enhancement as input data and an execution command to display a high image quality image and an execution command to activate the button for giving an instruction to display a high image quality image as ground truth data. It will be understood that the training data may be any training data where the instruction content indicated by the character data, voice data, or the like and the content of the execution command(s) correspond to each other, for example. Voice data may be converted into character data by using an acoustic model, a language model, and the like. Processing for reducing noise data superposed on the voice data using waveform data obtained by a plurality of microphones may be performed. The information processing apparatus may be configured such that character, voice, or other instructions and instructions given using a mouse, touch panel, and the like are selectable based on the examiner's instructions. The information processing apparatus may be configured such that whether to turn character, voice, or other instructions on or off can be selected based on the examiner's instructions.

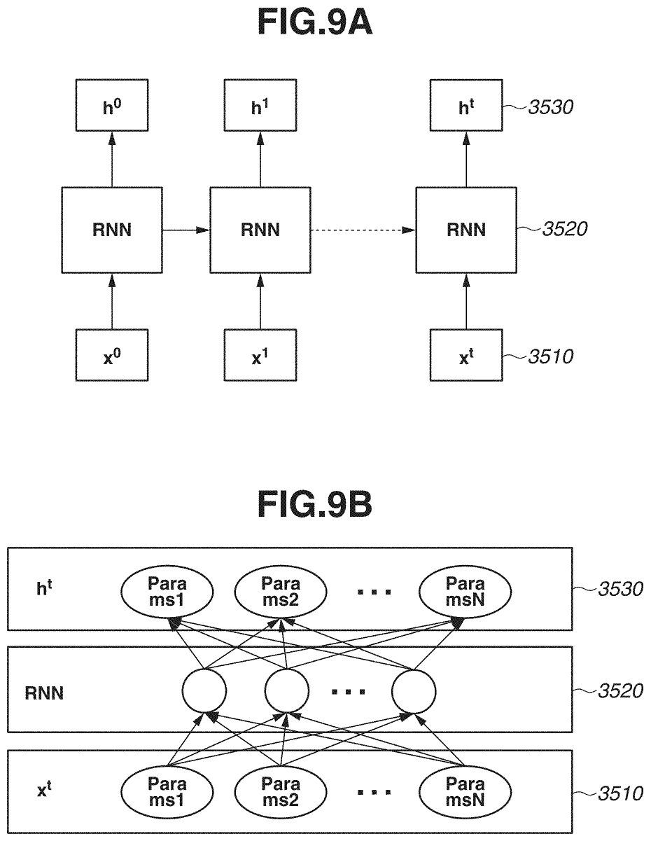

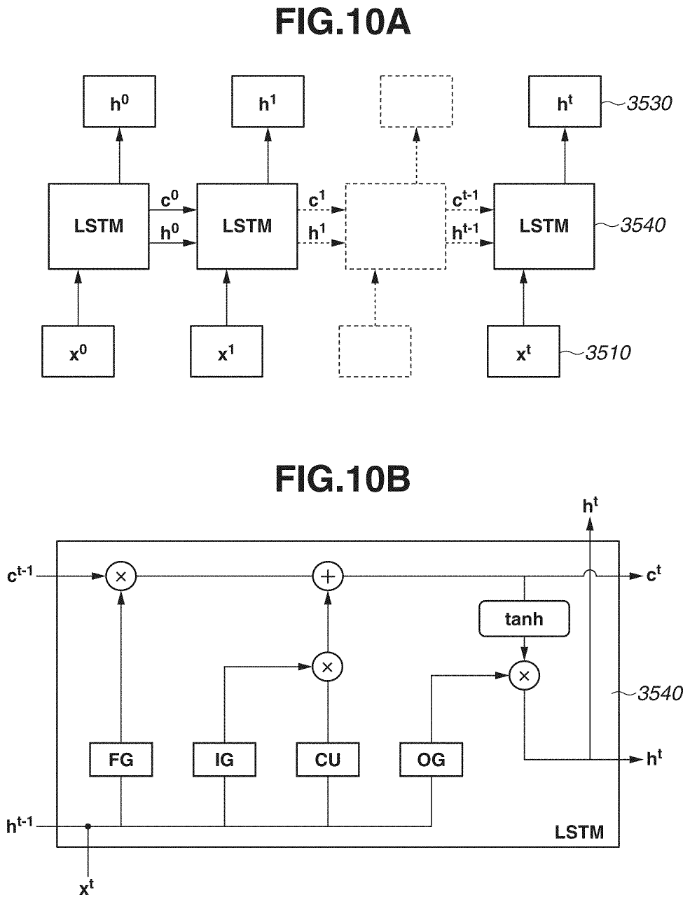

Here, machine learning includes the foregoing deep learning, and a recurrent neural network (RNN) can be used for at least part of a multilevel neural network, for example. An RNN that is a neural network for handling time series information will now be described as an example of a machine learning engine related to the present modification with reference to FIGS. 9A and 9B. Long short-term memory (LSTM), a kind of RNN, will be described with reference to FIGS. 10A and 10B.

FIG. 9A illustrates a structure of an RNN that is a machine learning engine. An RNN 3520 has a looped network structure, and inputs data x^{t} 3510 and outputs data h^{t} 3530 at time t. Since the RNN 3520 has a looped network function and can pass the state at the current time to the next state, the RNN 3520 can thus handle time series information. FIG. 9B illustrates an example of the input and output of parameter vectors at time t. The data x^{t} 3510 includes N pieces (Params1 to ParamsN) of data. The data h^{t} 3530 output from the RNN 3520 includes N pieces (Params1 to ParamsN) of data corresponding to the input data.

However, since the RNN is unable to handle long-term information during backpropagation, LSTM can sometimes be used. The LSTM includes a forget gate, an input gate, and an output gate, and can thus learn long-term information. FIG. 10A illustrates a structure of the LSTM. Information for the network, or LSTM 3540, to pass to the next time t is an internal state c^{t-1} of a network called cell, and output data h^{t-1}. The small letters in the diagram (c, h, and x) represent vectors.

Next, FIG. 10B illustrates details of the LSTM 3540. In FIG. 10B, a forget gate network FG, an input gate network IG, and an output gate network OG are a sigmoid layer each. The forget gate network FG, the input gate network IG, and the output gate network OG therefore output a vector the elements of which have a value of 0 to 1 each. The forget gate network FG determines how much past information to hold. The input gate network IG determines which values to update. A cell update candidate network CU is an activation function tanh layer. The cell update candidate network CU generates a new candidate vector to be added to the cell. The output gate network OG selects an element or elements of the cell candidate and selects how much information to pass to the next time.

The foregoing LSTM model is a basic form, and the networks described here are not restrictive. The connections between the networks may be changed. A quasi recurrent neural network (QRNN) may be used instead of the LSTM. Moreover, the machine learning engines are not limited to neural networks, and boosting, support vector machines, and the like may be used. If the examiner's instructions are input by using characters, voice, or the like, a technique related to natural language processing (such as Sequence to Sequence) may be applied. As a technique related to the natural language processing, a model that makes an output for each input sentence may be applied, for example. The foregoing various trained models are not limited to being applied to the examiner's instructions and may be applied to outputs to the examiner. An interaction engine (interaction model, trained model for interaction) that responds to the examiner with character, voice, or other output may be applied.

As a technique related to the natural language processing, a trained model obtained by performing unsupervised learning with document data in advance may be used. As a technique related to the natural language processing, a trained model obtained by further performing transfer learning (or fine tuning) on a trained model obtained by advance learning depending on the intended use may be used. As a technique related to the natural language processing, Bidirectional Encoder Representations from Transformers (BERT) may be applied, for example. As a technique related to the natural language processing, a model that can extract (express) context (feature amount) by itself by predicting specific words in text from the context both before and after may be applied. As a technique related to the natural language processing, a model that can determine a relationship (continuity) between two sequences (sentences) in input time-series data may be applied. As a technique related to the natural language processing, a model that uses a transformer encoder in a hidden layer and inputs and outputs a vector sequence may be applied.

Here, the examiner's instruction to which the present modification can be applied may be at least any one of instructions related to the following: switching of display of various images and analysis results, selection of the depth range for generating an en-face image, selection on use as training data for additional training, selection of a trained model, and output (such as display and transmission), storage, and the like of results obtained by using various trained models, described in the foregoing various exemplary embodiments and modifications. The examiner's instructions to which the present modification can be applied are not limited to instructions after imaging and may be instructions before imaging. Examples include instructions about various adjustments, instructions about settings of various imaging conditions, and instructions about the start of imaging. The examiner's instructions to which the present modification can be applied may be instructions about switching (screen transition) of the display screen.

The machine learning models may be ones combining an image-related machine learning model such as a CNN and a time series data-related machine learning model such as an RNN. Such a machine learning model can learn, for example, a relationship between a feature amount related to an image and a feature amount related to time series data. If the input layer of the machine learning model is a CNN and the output layer is an RNN, training may be performed using training data including medical images as input data and text related to the medical images (for example, the presence or absence of a lesion, the type of lesion, a recommendation for the next examination, etc.) as output data, for example. This enables, for example, even an examiner without much medical experience to easily figure out medical information about a medical image since medical information related to the medical image is automatically described in a text form. If the input layer of the machine learning model is an RNN and the output layer is a CNN, training may be performed using training data including medical text about lesions, observations, diagnoses, and the like as input data and medical images corresponding to the medical text as output data. This enables, for example, the examiner to easily search for medical images related to the case to be observed.

A machine translation engine (machine translation model, trained model for machine translation) for machine-translating character, voice, and other text into a given language may be used for instructions from the examiner and outputs to the examiner.

The information processing apparatus may be configured such that the given language can be selected based on the examiner's instructions. The foregoing techniques related to the natural language processing (for example, Sequence to Sequence) may be applied to the machine translation engine, for example. The information processing apparatus may be configured such that after the text input to the machine translation engine is machine-translated, the machine-translated text is input to the character recognition engine or the like, for example. The information processing apparatus may be configured such that text output from the foregoing various trained models is input to the machine translation engine, and text output from the machine translation engine is output, for example.

The foregoing various trained models may be used in combination. For example, the information processing apparatus may be configured such that characters corresponding to the examiner's instructions are input to the character recognition engine, and voice obtained from the input characters is input into another type of machine learning engine (such as the machine translation engine). For example, the information processing apparatus may be configured such that characters output from another type of machine learning engine are input to the character recognition engine, and voice obtained from the input characters is output. For example, the information processing apparatus may be configured such that voice corresponding to the examiner's instructions is input to the voice recognition engine, and characters obtained from the input voice are input into another type of machine learning engine (such as the machine translation engine). For example, the information processing apparatus may be configured such that voice output from another type of machine learning engine is input to the voice recognition engine, and characters obtained from the input voice are displayed on the display unit. Here, the information processing apparatus may be configured such that which to output to the examiner, the character output or the voice output, can be selected based on the examiner's instructions. The information processing apparatus may also be configured such that which to use as the examiner's instructions, the character input or the voice input, can be selected based on the examiner's instructions. The foregoing various configurations may be employed based on a selection made by the examiner's instructions.

### SEVENTH MODIFICATION

In the foregoing various exemplary embodiments and modification, high image quality images and the like may be stored into the storage unit based on the examiner's instructions. In registering a filename after the examiner's instructions to store a high image quality image or the like, a filename including information (for example, characters) indicating that the image has been generated by processing using the trained model for image quality enhancement (image quality enhancement processing) at a part (for example, at the beginning or at the end) of the filename may be displayed as a recommended filename in a state of being capable of editing based on the examiner's instructions. In displaying a high image quality image on various display screens such as report screens on the display unit, an indication that the displayed image is a high image quality image generated by the processing using the trained model for image quality enhancement may be displayed along with the high image quality image. In such a case, the user can easily identify, from the indication, that the displayed high image quality image is not the image itself obtained by imaging. This can reduce wrong diagnoses and improve diagnostic efficiency. The indication of being a high image quality image generated by the processing using the trained model for image quality enhancement may be any mode of indication from which the input image and the high image quality image generated by the processing can be identified. Not only the result of the processing using the trained model for image quality enhancement but the results of the processing using the foregoing various trained models may also be displayed with an indication that the results have been generated by the processing using those types of trained models.

Here, the display screen such as a report screen may be stored into the storage unit as image data based on the examiner's instructions. For example, a report screen may be stored into the storage unit as an image in which high image quality images and the like and indications of the images being high image quality images generated by the processing using the trained model for image quality enhancement are arranged in rows. As the indication of being a high image quality image generated by the processing using the trained model for image quality enhancement, an indication of what training data the trained model for image quality enhancement has been trained with may be displayed on the display unit. Such an indication may include a description of the types of input data and ground truth data in the training data, as well as any indication related to the input data and the ground truth data, like imaging regions included in the input data and the ground truth data. Here, not only for the processing using the trained model for image quality enhancement but also for the processing using the foregoing various trained models, the indication of what training data the types of trained models have been trained with may be displayed on the display unit.

The information processing apparatus may be configured such that information (for example, characters) indicating that an image has been generated by the processing using the trained model for image quality enhancement is displayed or stored in a state of being superimposed on the high image quality image or the like. Here, the superimposing position on the image may be located in any area (for example, in the corner of the image) not overlapping the area where the region of interest or the like to be imaged is displayed. The information processing apparatus may determine such a nonoverlapping area and superimpose the information on the determined area.

The information processing apparatus may be configured such that if the image quality enhancement button is set to be activated on the initial display screen of the report screen (the image quality enhancement processing is on) by default, a report image corresponding to the report screen including high image quality images and the like is transmitted to a server, such as an external storage unit, based on the examiner's instructions. The information processing apparatus may be configured such that if the image quality enhancement button is set to be activated by default, the report image corresponding to the report screen including high image quality images and the like is (automatically) transmitted to the server at the end of examination (for example, when the imaging observation screen or the preview screen is switched to the report screen based on the examiner's instructions). Here, the information processing apparatus may be configured such that a report image generated based on various default settings (for example, settings related to at least one of the following: the depth range for generating an en-face image on the initial display screen of the report screen, the presence or absence of a superimposed analysis map, whether an image is a high image quality image, and whether the screen is a display screen for follow-up) is transmitted to the server.

### EIGHTH MODIFICATION

In the foregoing various exemplary embodiments and modifications, an image (for example, a high image quality image, an image indicating an analysis result such as an analysis map, an image indicating an object recognition result, or an image indicating a segmentation result) obtained by a first type of trained model among the foregoing various trained models may be input to a second type of trained model different from the first type. Here, the information processing apparatus may be configured to generate the result of the processing by the second type of trained model (such as an analysis result, a diagnostic result, an object recognition result, or a segmentation result).

An image to be input into a second type of trained model different from a first type of trained model among the foregoing various trained models may be generated from an image input to the first type of trained model by using the result of the processing by the first type of trained model (such as an analysis result, a diagnostic result, an object recognition result, or a segmentation result). Here, the generated image is likely to be suitable as an image for the second type of trained model to process. The accuracy of the image obtained by inputting the generated image into the second type of trained model (for example, a high image quality image, an image indicating an analysis result such as an analysis map, an image indicating an object recognition result, or an image indicating a segmentation result) can thus be improved. The information processing apparatus may be configured to input a common image into the first type of trained model and the second type of trained model to generate (or display) processing results using the trained models. Here, for example, the information processing apparatus may be configured to generate (or display) the results of the processing using the trained models at the same time (in an interlocking manner) based on the examiner's instructions. The information processing apparatus may be configured such that the type of image to be input (for example, a high image quality image, an object recognition result, a segmentation result, or a similar case image), the type of processing result to be generated (or displayed) (for example, a high image quality image, a diagnostic result, an analysis result, an object recognition result, a segmentation result, or a similar case image), the type of input and the type of output (such as characters, voice, and language), and the like can each be selected based on the examiner's instructions. Here, the information processing apparatus may be configured such that at least one trained model is selected depending on the selected types. If a plurality of trained models is selected here, the way of combination of the plurality of trained models (such as the order of input of data) may be determined based on the selected types. The information processing apparatus may be configured, for example, such that the type of image to be input and the type of processing result to be generated (or displayed) can be selected to be different. The information processing apparatus may be configured to output information for prompting different selections to the examiner if the same types are selected. The trained models may be executed in any location. For example, some of the plurality of trained models may be used by a cloud server while the others may be configured to be used by a different server such as a fog server or an edge server.

The foregoing various trained models may be ones obtained by training with training data including two-dimensional medical images of test subjects, or ones obtained by training with training data including three-dimensional medical images of test subjects.

A similar case image search using an external database stored in a server or the like may be performed with an analysis result, a diagnostic result, and the like of the processing by the foregoing various trained models as a search key. The similar case image search using the external database stored in the server or the like may also be performed with an object recognition result, a segmentation result, and the like of the processing by the foregoing various trained models as a search key. In cases such as when a plurality of images stored in the database is managed with feature amounts of the respective plurality of images already attached as accessory information by machine learning and the like, a similar case image search engine (similar case image search model, trained model for similar case image search) using an image itself as a search key may be used. For example, the information processing apparatus can search various medical images for a similar case image related to a medical image by using the trained model for similar case image search (different from the trained model for image quality enhancement). For example, the display control unit 103 can display the similar case image obtained from the various medical images by using the trained model for similar case image search on the display unit. Here, the similar case image is an image having a feature amount similar to that of the medical image input to the trained model, for example. If, for example, the medical image input to the trained model includes a partial area such as an abnormal region, the similar case image is an image having a feature amount similar to that of the partial area such as an abnormal region. This not only enables efficient training for an accurate search of a similar case image, but also enables the examiner to efficiently make a diagnosis of an abnormal region if the medical image includes the abnormal region, for example. Moreover, a plurality of similar case images may be searched for, and the plurality of similar case images may be displayed such that the order of similarity of the feature amounts is identifiable. The information processing apparatus may be configured to additionally train the trained model for similar case image search by using training data including an image selected from a plurality of similar case images based on the examiner's instructions and the feature amount of the image. (Ninth Modification)

The processing for generating motion contrast data in the foregoing various exemplary embodiments and modifications is not limited to the configuration where the processing is performed based on the luminance values of tomographic images. Various types of processing may be applied to the interference signal obtained by the optical coherence tomography (OCT), the interference signal to which the Fourier transform is applied, the signal to which given processing is applied, and tomographic data including tomographic images and the like based on these signals. Similar effects can be provided even in such cases. For example, while a fiber optical system using a photocoupler is used as a splitting unit, a spatial optical system including a collimator and a beam splitter may be used. The OCT may be configured such that some of the components included in the OCT are separate from the OCT. A Michelson interferometer configuration may be 10201526US01 used for the OCT interference optical system. A Mach-Zehnder interferometer configuration may be used. The OCT may be a spectral domain OCT (SD-OCT) using a superluminescent diode (SLD) as a light source. The OCT may be any other type of OCT, like a swept-source OCT (SS-OCT) using a wavelength-swept light source capable of sweeping the wavelength of the emission light. Moreover, the present invention can also be applied to a line-OCT device (or SS-line-OCT device) using line light. The present invention can also be applied to a full field-OCT device (or SS-full field-OCT device) using area light. While the information processing apparatus obtains the interference signal obtained by the OCT and the three-dimensional tomographic images and the like generated by the information processing apparatus, the configuration for the information processing apparatus to obtain such signals and images are not limited thereto. For example, the information processing apparatus may obtain such signals and images from a server or an imaging device connected via a local area network (LAN), a wide area network (WAN), the Internet, and/or the like.

The trained models can be located in the information processing apparatus. For example, the trained models can be constituted by software modules executed by a processor such as a CPU. Alternatively, the trained models may be located in another server or the like connected to the information processing apparatus. In such a case, the information processing apparatus can perform image quality enhancement processing using a trained model by connecting to the server including the trained model via a given network such as the Internet. (Tenth Modification)

Medical images to be processed by the information processing apparatus (medical image processing apparatus) or the information processing method (medical image processing method) according to the foregoing various exemplary embodiments 10201526US01 and modifications may include images obtained by using any modality (imaging device, imaging method). The medical images to be processed can include medical images obtained by a given imaging device and the like, and images generated by the medical image processing apparatus or the medical image processing method.

The images to be processed further include an image of a predetermined region of the examinee (test subject), and the image of the predetermined region includes at least part of the predetermined region of the examinee The medical image may include other regions of the examinee. The medical images may be still images or moving images, and may be monochrome images or color images. The medical images may be ones showing a structure (shape) of a predetermined region or images showing a function thereof. Examples of the images showing a function include images showing hemodynamics (blood flow, blood flow rate, and the like), such as an OCTA image, a Doppler OCT image, a functional magnetic resonance imaging (fMRI) image, and an ultrasonic Doppler image. The predetermined region of the examinee may be determined based on the imaging target, and may include organs such as a human eye (eye to be examined), brain, lungs, intestines, heart, pancreas, kidney, and liver, and regions such as the head, breast, legs, and arms.

The medical images may be tomographic images or front images of the examinee Examples of the front images include a fundus front image, a front image of the anterior eye part, a fundus image obtained by fluorescence imaging, and an en-face image generated using data on at least part of the range of data obtained by the OCT (three-dimensional OCT data) in the depth direction of the imaging target. The en-face image may be an OCTA en-face image (motion contrast front image) generated using at least part of the range of three-dimensional OCTA data (three-dimensional motion contrast data) in the depth direction of the imaging target. Three-dimensional OCT data and 10201526US01 three-dimensional motion contrast data are examples of three-dimensional medical image data.

As employed herein, motion contrast data refers to data indicating a change between a plurality of pieces of volume data obtained by controlling the measurement light to scan the same area (same positions) of an eye to be examined a plurality of times. Here, the volume data includes a plurality of tomographic images obtained at different positions. The motion contrast data can be obtained as volume data by obtaining data indicating a change between a plurality of tomographic images obtained at substantially the same positions at each of the different positions. A motion contrast front image is also referred to as an OCTA front image (OCTA en-face image) related to OCT angiography (OCTA) for measuring the motion of a blood flow. Motion contrast data is also referred to as OCTA data. Motion contrast data can be determined, for example, as decorrelation values, variance values, or maximum values divided by minimum values (maximum values/minimum values) between two tomographic images or corresponding interference signals, and may be determined by any conventional method. Here, the two tomographic images can be obtained by controlling the measurement light to scan the same area (same positions) of the eye to be examined a plurality of times, for example.

An en-face image is a front image generated by projecting data in the range between two layer boundaries upon the XY plane, for example. Here, the front image is generated by projecting on a two-dimensional plane the data corresponding to a depth range that is at least part of the depth range of volume data (three-dimensional tomographic image) obtained using optical interference and is determined based on two reference planes, or integrating the data. The en-face image is a front image generated by projecting the data corresponding to the depth range determined based on detected retinal layers in the volume data upon the two-dimensional plane. For example, the data corresponding to the depth range determined based on the two reference planes can be projected upon the two-dimensional plane, for example, by using a technique for using representative values of the data in the depth range as pixels values on the two-dimensional plane. Here, the representative values can include values such as averages, medians, and maximum values of the pixel values in the depth-wise range of the area surrounded with the two reference planes. An example of the depth range related to the en-face image may be a range including a predetermined number of pixels in a deeper direction or a shallower direction than either one of the two layer boundaries related to the detected retinal layers. An example of the depth range related to the en-face image may be a range obtained by modifying (offsetting) the range between the two layer boundaries related to the detected retinal layers based on the operator's instructions.

The imaging devices are devices for capturing images for use in diagnosis. Examples of the imaging devices include devices that obtain an image of a predetermined region of the examinee by irradiating the predetermined region with light, radiations such as X-rays, electromagnetic waves, ultrasonic waves, and the like, and devices that obtain an image of a predetermined region by detecting radiations emitted from the object. More specifically, the imaging devices according to the foregoing various exemplary embodiments and modifications include at least an X-ray imaging device, a computed tomography (CT) device, a magnetic resonance imaging (MRI) device, a positron emission tomography (PET) device, a single photon emission computed tomography (SPECT) device, an SLO device, an OCT device, an OCTA device, a fundus camera, and/or an endoscope.

OCT devices may include time-domain OCT (TD-OCT) devices and Fourier-domain OCT (FD-OCT) devices. Fourier-domain OCT devices may include a spectral-domain OCT (SD-OCT) device and a swept source OCT (SS-OCT) device. OCT devices may also include a Doppler-OCT device. SLO devices and OCT devices may include an adaptive optics SLO (AO-SLO) device and an adaptive optics OCT (AO-OCT) device using an adaptive optical system. SLO devices and OCT devices may also include a polarization-sensitive SLO (PS-SLO) device and a polarization-sensitive OCT (PS-OCT) device for visualizing information about a polarization phase difference or depolarization. SLO devices and OCT devices may include a pathological microscope SLO device and a pathological microscope OCT device. SLO devices and OCT devices may include a handheld SLO device and a handheld OCT device. SLO devices and OCT devices may include a catheter SLO device and a catheter OCT device.

### Other Exemplary Embodiments

The present invention is also implemented by performing the following processing. That is, the processing includes supplying software (program) for implementing one or more functions of the foregoing various exemplary embodiments and modifications to a system or an apparatus via a network or various storage media, and reading and executing the program by a computer (or CPU, MPU, or the like) of the system or apparatus.

The present invention can also be implemented by supplying software (program) for implementing one or more functions of the foregoing various exemplary embodiments and modifications to a system or an apparatus via a network or various storage media, and reading and executing the program by a computer of the system or apparatus. The computer includes one or a plurality of processors or circuits, and can include a plurality of separate computers or a network of a plurality of separate processors or circuits to read and execute computer-executable instructions.

Here, the processors or circuits can include a central processing unit (CPU), a micro processing unit (MPU), a graphics processing unit (GPU), an application specific integrated circuit (ASIC), or a field-programmable gate array (FPGA). The processors or circuits can also include a digital signal processor (DSP), a data flow processor (DFP), or a neural processing unit (NPU).

The present invention is not limited to the above embodiments and various changes and modifications can be made within the spirit and scope of the present invention. Therefore, to apprise the public of the scope of the present invention, the following claims are made.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to one of the disclosed techniques, settings related to different types of imaging data can be individually set.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus comprising:
a storage unit configured to store information individually set for each of a plurality of different types of imaging as transmission settings for a plurality of pieces of imaging data obtained by the plurality of different types of imaging; and
a transmission unit configured to transmit imaging data on a test subject based on the stored information, the imaging data being obtained by any one of the plurality of different types of imaging.

2. The information processing apparatus according to claim 1, wherein the transmission unit is configured to, in a case where information that automatic transmission is set to on in the transmission setting for the one type of imaging is stored, transmit the imaging data obtained by the one type of imaging based on the stored information with an examiner's instruction to switch an imaging screen for performing the one type of imaging to another display screen as a trigger to start transmission.

3. The information processing apparatus according to claim 1, wherein the transmission unit is configured to, in a case where information that transmission of a report image is set to on as the transmission setting for the one type of imaging is stored and image quality enhancement processing is on as an initial display setting of a report screen, transmit, as the imaging data, a report image corresponding to a report screen displaying a second medical image obtained by performing the image quality enhancement processing on a first medical image obtained by the one type of imaging.

4. The information processing apparatus according to claim 3, wherein the image quality enhancement processing is processing for generating the second medical image from the first medical image by using a trained model obtained by training with a medical image of a test subject.

5. The information processing apparatus according to claim 3, wherein the transmission settings are configured such that a transmission setting for data including the first medical image and the second medical image as a set is includable.

6. The information processing apparatus according to claim 5, wherein the data including the set is training data for additional training.

7. The information processing apparatus according to claim 1, wherein a plurality of ophthalmologic imaging devices configured to perform the plurality of different types of imaging on an eye to be examined of the test subject includes a fundus camera and an optical coherence tomography (OCT) device.

8. The information processing apparatus according to claim 1,
wherein the transmission settings are configured such that a plurality of patterns is registrable, and
wherein the transmission unit is configured to transmit the test subject's imaging data obtained by the one type of imaging based on the stored information in order of the plurality of registered patterns.

9. The information processing apparatus according to claim 1, wherein the transmission settings are configured to be individually settable for the respective plurality of different types of imaging based on an instruction from the examiner.

10. The information processing apparatus according to claim 1, wherein the transmission settings include a transmission content, a transmission type, and a transmission destination as common settings, and an image size and a presence or absence of automatic transmission as individual settings.

11. The information processing apparatus according to claim 1, further comprising a display control unit configured to display the imaging data on a display unit.

12. An information processing apparatus comprising:
a display control unit configured to display a second medical image having higher image quality than a first medical image of a test subject on a display unit, the first medical image being obtained by any one of a plurality of different types of imaging, the second medical image being generated from the first medical image by using a trained model obtained by training with a medical image of a test subject; and
a transmission unit configured to transmit a report image corresponding to a report screen displaying the second medical image with an examiner's instruction as a trigger to start transmission.

13. The information processing apparatus according to claim 12, wherein the display control unit is configured to display an optical coherence tomography angiography (OCTA) front image generated as the second medical image and an OCT tomographic image generated as the second medical image on the display unit, a line indicating a position of the OCT tomographic image being superimposed on the OCTA front image, the OCT tomographic image corresponding to a position of the line moved on the OCTA front image based on an instruction from the examiner.

14. The information processing apparatus according to claim 13, wherein the display control unit is configured to superimpose information on the OCT tomographic image corresponding to the position of the line, the information indicating a blood vessel region in an OCTA tomographic image corresponding to the position of the line, the OCTA tomographic image being generated as the second medical image.

15. The information processing apparatus according to claim 12, wherein a filename of the second medical image generated by using the trained model includes information in a state of being editable based on an instruction from the examiner, the information indicating that the image is generated by performing image quality enhancement processing.

16. The information processing apparatus according to claim 12, wherein input of a medical image other than training data into the trained model under additional training is disabled, and input of the medical image other than the training data into a backup trained model that is a trained model identical to the trained model before execution of the additional training is executable.

17. The information processing apparatus according to claim 16, wherein the display control unit is configured to display a comparison result or a determination result about whether the comparison result falls within a predetermined range on the display unit, the comparison result being obtained by using an image obtained using the trained model after the execution of the additional training and an image obtained using the trained model before the execution of the additional training.

18. The information processing apparatus according to claim 11, wherein the display control unit is configured to display at least one of (a) an analysis result related to a medical image obtained by the one type of imaging on the display unit, the analysis result being generated using a trained model for analysis result generation obtained by training with a medical image of a test subject, (b) a diagnostic result related to a medical image obtained by the one type of imaging on the display unit, the diagnostic result being generated using a trained model for diagnostic result generation obtained by training with a medical image of a test subject, (c) as information about an abnormal region, information about a difference between (i) a medical image obtained by the one type of imaging and (ii) an image obtained by input of the medical image to a generative adversarial network or an auto-encoder on the display unit, (d) a similar case image related to a medical image obtained by the one type of imaging on the display unit, the similar case image being searched for by using a trained model for similar case image search obtained by training with a medical image of a test subject, and (e) an object recognition result or a segmentation result related to a medical image obtained by the one type of imaging on the display unit, the object recognition result or the segmentation result being generated using a trained model for object recognition or a trained model for segmentation obtained by training with a medical image of a test subject.

19. The information processing apparatus according to claim 11, wherein the display control unit is configured to display an image, information, or a result obtained by inputting a plurality of medical images obtained by the plurality of different types of imaging into a trained model on the display unit.

20. The information processing apparatus according to claim 1, wherein an examiner's instruction about a trigger for the transmission unit to start transmission is information obtained by using at least one trained model among a trained model for character recognition, a trained model for voice recognition, and a trained model for gesture recognition.

21. An information processing method comprising:
storing information individually set for each of a plurality of different types of imaging as transmission settings for a plurality of pieces of imaging data obtained by the plurality of different types of imaging; and
transmitting imaging data on a test subject based on the stored information, the imaging data being obtained by any one of the plurality of different types of imaging.

22. An information processing method comprising:
displaying a second medical image having higher image quality than a first medical image of a test subject on a display unit, the first medical image being obtained by any one of a plurality of different types of imaging, the second medical image being generated from the first medical image by using a trained model obtained by training with a medical image of a test subject; and
transmitting a report image corresponding to a report screen displaying the second medical image with an examiner's instruction as a trigger to start transmission.

23. A non-transitory computer-readable storage medium storing a program for causing a computer to execute each method of the information processing method according to claim 21.

24. A non-transitory computer-readable storage medium storing a program for causing a computer to execute each method of the information processing method according to claim 22.
